SEQUENCE LISTING (23 sequences)

SEQ ID NO: 1 - protein, 9 aa, Human
KLPNFGFVV

SEQ ID NO: 2 - protein, 9 aa, human
IMFRGEVRL

SEQ ID NO: 3 - protein, 21 aa, mouse
SATPPPAEPASLPQEPPKPRV

SEQ ID NO: 4 - DNA, 1975 bp, human
  misc_feature (1897)..(1897): n = a, c, g or t
gattttatgagtatttcttccagggtaaaaagcaaaagaattccgggcccgggaagaggtggttgtgaggcagacgaactcgcggctctccggcttccgaggcttccgagttgtcggaggaagggggcggcgagcaataagaacccgccgcacccggtcctcagcgactcttctgacctccgcgsgacgtacccgccgccgccgttggctggagcatttgacattgtgcagcaaagaaatggttatggagaagcccagtccgctgcttgtagggcgggagtttgtgaggcaatattatactttgctgaataaagctccggaatatttacacaggttttatggcaggaattcttcctatgttcatggtggagtagatgctagtggaaagccccaggaagctgtttatggccaaaatgatatacaccacaaagtattatctctgaacttcagtgaatgtcatactaaaattcgtcatgtggatgctcatgcaaccttgagtgatggagtagttgtccaggtcatgggtttgctgtctaacagtggacaaccagaaagaaagtttatgcaaacctttgttctggctcctgaaggatctgttccaaataaattttatgttcacaatgatatgtttcgttatgaagatgaagtgtttggtgattctgagcctgaacttgatgaagaatcagaagatgaagtagaagaggaacaagaagaaagacaaccatctcctgaacctgtgcaagaaaatgctaacagtggttactatgaagctcaccctgtgactaatggcatagaggagcctttggaagaatcctctcatgaacctgaacctgagccagaatctgaaacaaagactgaagagctgaaaccacaagtggaggagaagaacttagaagaactagaggagaaatctactactcctcctccggcagaacctgtttctctgccacaagaaccaccaaaggctttctcctgggcttcagtgaccagtaaaaacctgcctcctagtggtactgtttcttcctctggaattccatcccatgttaaagcaccagtctcacagccaagagtcgaagctaaaccagaagttcaatctcagccacctcgtgtgcgtgaacaacgacctagagaacgacctggttttcctcctagaggaccaagaccaggcagaggagatatggaacagaatgactctgacaaccgtagaataattcgctatccagatagtcatcaactttttgttggtaacttgccacatgatattgatgaaaatgagctaaaggaattcttcatgagttttggaaacgttgtggaacttcgcatcaataccaagggtgttgggggaaagcttccaaattttggttttgtggtttttgatgactctgaaccagttcagagaatcttaattgcaaaaccgattatgtttcgaggggaagtacgtttaaatgtggaagagaaaaaaacaagagctgcaagagagcgagaaaccagaggtggtggtgatgatcgcagggatattaggcgcaatgatcgaggtcccggtggtccacgtggaattgtgggtggtggaatgatgcgtgatcgtgatggaagaggacctcctccaaggggtggcatggcacagaaacttggctctggaagaggaaccgggcaaatggagggccgcttcacaggacagcgtcgctgaagctccactgttggcaaagtcttggcagtggtacattattcatcgtgtttgcattcttgttaattttttttttggctttggaatgtgacacagcctttttgatcatttctttgatgtgaaaagcatctttggttatcagttaaattgaggtggacattatttccccaatttcacaacaggattcacattgttaatttataaatctagacntggagaattaaggactgagaaatgaccatatcttaaactatctacgacaaagtgaacttaaaaggacatgcccactga

SEQ ID NO: 5 - protein, 482 aa, Human [sequence omitted]

SEQ ID NO: 6 - protein, 61 aa, Unknown
  Variant
VRAILPYTKVPDTDEISFLKGDMFIVHNELEDGWMWVTNLRTDEQGLIVEDLVEEVGREED

SEQ ID NO: 7 - protein, 482 aa, Mouse
MVMEKPSPLLVGREFVRQYYTLLNKAPEYLHRFYGRNSSYVHGGVDASGKPQEAVYGQNDIHHKVLSLNFSECHTKIRHVDAHATLSDGVVVQVMGLLSNSGQPERKFMQTFVLAPEGSVPNKFYVHNDMFRYEDEVFGDSEPELDEESEDEVEEEQEDRQPSPEPVQENANSAYYDAHPVTNGIEEPLEESSHEPEPEPESETKTEELKPQVEEKHLEELEEKSATPPPAEPASLPQEPPKAFSWASVTSKNLPPSGTVSSSGIPPHVKAPVSQPRVDAKPEVQSQPPRVREQRPRERPGFPPRGPRPGRGDMEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMTQKLGSGRGTGQMEGRFTGQRR

SEQ ID NO: 8 - protein, 449 aa, Mouse
MVMEKPSPLLVGREFVRQYYTLLNKAPEYLHRFYGRNSSYVHGGVDASGKPQEAVYGQNDIHHKVLSLNFSECHTKIRHVDAHATLSDGVVVQVMGLLSNSGQPERKFMQTFVLAPEGSVPNKFYVHNDMFRYEDEVFGDSEPELDEESEDEVEEEQEDRQPSPEPVQENANSAYYDAHPVTNGIEEPLEESSHEPEPEPESETKTEELKPQVEEKHLEELEEKSATPPPAEPASLPQEPPKPRVDAKPEVQSQPPRVREQRPRERPGFPPRGPRPGRGDMEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMTQKLGSGRGTGQMEGRFTGQRR

SEQ ID NO: 9 - protein, 449 aa, Human
MVMEKPSPLLVGREFVRQYYTLLNKAPEYLHRFYGRNSSYVHGGVDASGKPQEAVYGQNDIHHKVLSLNFSECHTKIRHVDAHATLSDGVVVQVMGLLSNSGQPERKFMQTFVLAPEGSVPNKFYVHNDMFRYEDEVFGDSEPELDEESEDEVEEEQEERQPSPEPVQENANSGYYEAHPVTNGIEEPLEESSHEPEPEPESETKTEELKPQVEEKNLEELEEKSTTPPPAEPVSLPQEPPKPRVEAKPEVQSQPPRVREQRPRERPGFPPRGPRPGRGDMEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMAQKLGSGRGTGQMEGRFTGQRR

SEQ ID NO: 10 - protein, 466 aa, Human
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQ

SEQ ID NO: 11 - protein, 465 aa, Mouse
MVMEKPSPLLVGREFVRQYYTLLNQAPDMLHRFYGKNSSYAHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQTVSNDLEEHLEEPVVEPEPEPEPEPEPEPVSDIQEDKPEAALEEAAPDDVQKSTSPAPADVAPAQEDLRTFSWASVTSKNLPPSGAVPVTGTPPHVVKVPASQPRPESKPDSQIPPQRPQRDQRVREQRINIPPQRGPRPIREAGEPGDVEPRRMVRHPDSHQLFIGNLPHEVDKSELKDFFQNFGNVVELRINSGGKLPNFGFVVFDDSEPVQKVLSNRPIMFRGAVRLNVEEKKTRAAREGDRRDNRLRGPGGPRGGPSGGMRGPPRGGMVQKPGFGVGRGITTPRQ

SEQ ID NO: 12 - DNA, 21 bp, Artificial sequence
  Primer
ggaggcatggtgcagaaacca

SEQ ID NO: 13 - DNA, 21 bp, Artificial sequence
  Primer
caggaaagggaagagagggag

SEQ ID NO: 14 - DNA, 21 bp, Artificial sequence
  Primer
gtcttggcagtggtacattat

SEQ ID NO: 15 - DNA, 27 bp, Artificial sequence
  Primer
agttcactttgtcgtagatagtttaag

SEQ ID NO: 16 - DNA, 32 bp, Artificial sequence
  Primer
atggtgatggagaagcctagtcccctgctggt

SEQ ID NO: 17 - DNA, 24 bp, Artificial sequence
  Primer
atggttatggagaagcccagtccg

SEQ ID NO: 18 - DNA, 24 bp, Artificial sequence
  Primer
atcaagttcaggctcagaatcacc

SEQ ID NO: 19 - DNA, 24 bp, Artificial sequence
  Primer
ctgaggctcagtgacaaacccaac

SEQ ID NO: 20 - DNA, 26 bp, Artificial sequence
  Primer
gcttcagcgacgctgtcctgtgaagc

SEQ ID NO: 21 - DNA, 24 bp, Artificial sequence
  Primer
ctgccgtggcgcaagcccccttcc

SEQ ID NO: 22 - protein, 146 aa, human
MVMEKPSPLLVGREFVRQYYTLLNKAPEYLHRFYGRNSSYVHGGVDASGKPQEAVYGQNDIHHKVLSLNFSECHTKIRHVDAHATLSDGVVVQVMGLLSNSGQPERKFMQTFVLAPEGSVPNKFYVHNDMFRYEDEVFGDSEPELD

SEQ ID NO: 23 - DNA, 438 bp, human
atggttatggagaagcccagtccgctgcttgtagggcgggagtttgtgaggcaatattatactttgctgaataaagctccggaatatttacacaggttttatggcaggaattcttcctatgttcatggtggagtagatgctagtggaaagccccaggaagctgtttatggccaaaatgatatacaccacaaagtattatctctgaacttcagtgaatgtcatactaaaattcgtcatgtggatgctcatgcaaccttgagtgatggagtagttgtccaggtcatgggtttgctgtctaacagtggacaaccagaaagaaagtttatgcaaacctttgttctggctcctgaaggatctgttccaaataaattttatgttcacaatgatatgtttcgttatgaagatgaagtgtttggtgattctgagcctgaacttgat

(19) United States
(12) Patent Application Publication
(10) Pub. No.: US 2006/0058505 A1
(43) Pub. Date: Mar. 16, 2006

(54) Nucleic acid and polypeptide linked to breast cancer and uses therefor

(76) Inventors: Derek Kennedy, Queensland (AU); Derek Hart, Queensland (AU); Juliet French, Queensland (AU); Jose Alejandro Lopez Ramirez, Queensland (AU)

Correspondence Address: MORRISON & FOERSTER LLP, 12531 HIGH BLUFF DRIVE, SUITE 100, SAN DIEGO, CA, 92130-2040, US

(21) Appl. No.: 10/487,466

(22) PCT Filed: Aug. 23, 2002
(86) PCT No.: PCT/AU02/01136

§ 371 Date: Oct. 4, 2004

(30) Foreign Application Priority Data
Aug. 23, 2001 (AU) PR 7211

(51) Int. Cl. C07K 14/82; C07H 21/04; C12P 21/06

(52) U.S. Cl. 530/350; 435/69.1; 435/320.1; 435/325; 536/23.5

(57) ABSTRACT

The present invention relates to an isolated domain of G3BP-2 that mediates binding between G3BP-2 and other proteins, and nucleic acids encoding same. The invention also relates to a method for diagnosing, treating and preventing breast cancer including the step of using a nucleic acid and/or encoded polypeptide for G3BP-2, or fragment thereof, to detect, treat or prevent breast cancer in a mammal, preferably human. In one particular form, the invention relates to an antigen presenting cell, preferably a dendritic cell, that is capable of presenting G3BP-2 or fragments thereof. The invention also relates to lymphocytes, in particular cytotoxic T-lymphocytes, that are G3BP-2 antigen specific.

### FIELD OF THE INVENTION

THIS INVENTION relates to an isolated domain of G3BP-2 that mediates binding between G3BP-2 and other proteins, and nucleic acids encoding same. More particularly, a method for diagnosing, treating and preventing breast cancer including the step of using a nucleic acid and/or encoded polypeptide for G3BP-2 to detect, treat or prevent breast cancer in a mammal.

### BACKGROUND OF THE INVENTION

ras-GTPase-Activating Protein SH3-Domain-binding Proteins (G3BPs) are a family of proteins which comprise SH3 domain-binding motifs which have been shown to specifically bind the ras-GTPase-activating protein, rasGAP¹²⁰ (Parker et al., 1996; Kennedy et al, 1997). Furthermore, this family of proteins have been shown to be RNA-binding proteins (Kennedy et al., 1997) that may have an RNAase activity on c-myc transcripts (Gallouzi et al., 1998). Both the ras-GAP signalling pathway and c-myc have been implicated in oncogenic activity (Bos, 1989; Facchini and Penn, 1998). The evidence presented suggests that the G3BP family of proteins are members of a novel signal transduction mechanism that utilises components of previously described pathways to regulate mRNA stability and through these pathways may regulate oncogenic signals or factors. These activities may be modulated through their SH3 domain-binding activity (Parker et al., 1996), their RNAase activity (Gallouzi et al., 1998) or their helicase activity (Costa et al., 1999). G3BPs have recently been shown to be upregulated at the transcriptional level in some cancers (Guitard et al., 2001).

rasGAP¹²⁰ is an important regulator of signal transduction (Pomerance et al., 1996) as it sits at the nexus of positive and negative control of the oncogene ras. rasGAP¹²⁰ itself stimulates the hydrolysis of GTP bound ras (reviewed in Tocque et al., 1997) and thereby regulates the activity of ras. The amino-terminus of rasGAP¹²⁰ comprises a Scr homology (SH3) domain (Tocque et al. 1997) which has been implicated in an effector-like activity (Duchesne et al., 1993).

Human G3BP-1 was first identified by its co-immunoprecipitation with rasGAP¹²⁰ using an antibody raised to the carboxy-terminal domain of rasGAP¹²⁰ (Parker et al., 1996). G3BP was the first protein shown to bind the rasGAP¹²⁰ SH3 domain, however, other rasGAP¹²⁰ SH3 binding proteins have since been reported, including a 14 kDa protein (Hu and Settleman 1997) and the huntingtin protein (Liu et al., 1997). Genetic studies in Drosophila support a role for G3BP in ras signaling (Pazman et al., 2000).

The inventors previously cloned and sequenced mouse G3BP-2 as part of a general screening for RNA Recognition motif (RRM)-containing proteins (Kennedy et al., 1997). Primary sequence analysis of G3BPs also indicated that they contain an RNA Recognition Motif (RRM) (Nagai et al., 1995), an RGG domain (Burd and Dreyfuss 1994; Siomi and Dreyfuss 1997) and a Nuclear Transport Factor 2-like (NTF2-like) domain (Suyama et al., 2000). The proposed structure of the RRM in G3BP has been reported elsewhere (Kennedy et al. 1997). The G3BPs also contain acid-rich and RGG domains which are often considered auxiliary domains for RRM-type RNA-binding proteins (Burd and Dreyfuss 1994; Siomi and Dreyfuss 1997). These structural motifs are consistent with a recent finding that G3BP-1 is implicated in RNA metabolism by acting in vitro as a cleavage factor for c-myc transcripts (Gallouzi et al. 1998).

NTF2 polypeptide is involved in nuclear transport of polypeptides and appears to be facilitated by binding RanGDP in the cytoplasm. Once NTF2/RanGDP is bound to a cargo the complex is imported to the nucleus where it is released and the Ran nucleotide exchange factor, RCC1, converts RanGDP to RanGTP. This signals export of NTF2 to the cytoplasm where RanGTP is hydrolysed by Ran GTPase activating protein (RanGAP) and the system is reset (reviewed in Macara 1999). The NTF2-like domain of G3BP-2 may target G3BP-2 to the nuclear envelope, although a mechanism for this activity is unclear (Prigent et al., 2000).

RNA processing is an integral part of cellular metabolism controlled through pre-mRNA splicing, RNA transport and RNA stability (Dreyfuss et al., 1996). Regulation of RNA metabolism has been shown to play an important role in development. Recently there has been increased interest in the control of mRNA translation mediated by RNA-binding proteins, in particular the role of these proteins in 5′ UTR interactions that influence elongation factors (Svitkin et al., 1996) as well as 3′ interactions involving translational activity (Dreyfuss et al. 1996) and degradation (Gallouzi et al., 1998). It is important to characterise the mechanisms that allow RNA-binding proteins to respond to environmental and developmental signals through transduction cascades in order to understand their role in human diseases.

SH3 domains were initially characterised in signal transduction proteins such as Src, Fyn and Grb as well as rasGAP¹²⁰. Typically these domains interact with proline rich motifs with a minimum consensus of PxxP (Urquhart et al., 2000 and papers cited therin). It has also been shown that the acidic and PxxP domains, and not the RNA-binding domain nor the NTF2-like domain of G3BP-2, are sufficient to mediate binding to IκBα (Prigent et al; 2000). Primary sequence analysis of alternatively spliced homologues of human G3BP-2a and G3BP-2b reveals that they respectively comprise five and six minimal potential SH3 domain-binding motifs (Lee et al., 1996). As G3BP-2a and G3BP-2b comprise PxxP sequences, it was predicted that these proline-rich motifs would bind with SH3 domains of polypeptides.

Major advances have been achieved in the early diagnosis (screening mammography) and treatment (adjuvant therapy) of breast cancer and this has translated into a significant reduction in the mortality generated by this disease (Chlebowski, 2002). However, breast cancer still accounts for 26% of the cancers diagnosed in Australia in 1999, and in 1996 there were 9,556 new cases diagnosed with 2,619 deaths ((AIHW), 1999). An additional important input into the better management of the disease has been the characterization of genetic predisposing markers BRCA1 and 2 (reviewed in (Nathanson et al., 2001)), allowing the prediction of a proportion (10%) of women at risk to develop breast cancer. Considering the reduced adjuvant therapeutic options currently available (Pritchard et al., 2002) and the risks involved with their use, novel strategies are urgently needed that could prevent the development of the disease at early stages of the disease and/or in those with higher genetic risk.

### SUMMARY OF THE INVENTION

Although G3BPs have been shown to bind the SH3 domain of GAP¹²⁰ the inventors were surprised to discover that this binding is mediated through the N-terminal NTF2-like domain of G3BP and not facilitated by a proline-rich motif (PxxP) contained within G3BP-2 as the prior art would suggest. The smallest G3BP truncated protein that was capable of binding to the SH3 domain of rasGAP¹²⁰ did not contain any of the predicted PxxP motifs normally associated with SH3 binding. The unexpected results clearly showed that the N-terminal NTF2-like domain of G3BP is responsible for the binding interactions with N-terminal rasGAP¹²⁰. This finding has led to novel uses of G3BP, in particular the NTF2-like domain thereof, as described herein for identifying and producing potential reagents for diagnosing, treating or preventing breast cancer.

In a first aspect, the invention provides an isolated G3BP-2 protein fragment comprising an NTF2-like domain, said isolated G3BP2 protein fragment capable of binding another protein by way of said NTF2-like domain.

Preferably, the G3BP-2 protein comprises G3BP-2a and/or G3BP-2b proteins.

Preferably, the another protein is selected from the group consisting of: ran nuclear pore polypeptide, ubiquitin hydrolase and GAP¹²⁰.

More preferably, the ubiquitin hydrolase is ODE1.

The NTF2-like domain preferably is encoded by amino acid residues 1 to 146 as set forth in SEQ ID NO: 22.

In a second aspect the invention provides an isolated protein complex comprising a G3BP-2 protein having an NTF2-like domain and another protein bound to the NTF2-like domain, selected from the group consisting of: ran nuclear pore polypeptide, ubiquitin hydrolase and GAP¹²⁰.

In a third aspect, the invention provides an isolated G3BP-2 protein, inclusive of a fragment, homolog, variant or derivative thereof capable of eliciting an immune response in an animal.

Preferably, the animal is human.

Preferably, the G3BP-2 fragment is selected from the group consisting of:

| | | |
|---|---|---|
| (i) | KLPNFGFW; | [SEQ ID NO:1] |
| (ii) | IMFRGEVRL; | [SEQ ID NO:2] |
| and | | |
| (iii) | SATPPPAEPASLPQEPPKPRV | [SEQ ID NO:3] |

In a fourth aspect, the invention provides an isolated G3BP-2 protein fragment selected from the group consisting of:

| | | |
|---|---|---|
| (a) | KLPNFGFW; | [SEQ ID NO:1] |
| (b) | IMFRGEVRL; | [SEQ ID NO:2] |
| and | | |
| (c) | SATPPPAEPASLPQEPPKPRV | [SEQ ID NO:3] |

In a fifth aspect, the invention provides an isolated nucleic acid encoding a protein of the first aspect, inclusive of fragments, homologs, variants and derivatives thereof, each capable of binding another protein by way of said NTF2-like domain.

In one form, the isolated nucleic acid encodes a protein comprising the NTF2-like domain comprising an amino acid sequence as set forth in SEQ ID NO: 22, said NTF2-like domain being encoded by amino acid residues 1 to 146, wherein amino acid residue 1 is the first methionine (M).

In another form, the isolated nucleic acid comprises a nucleotide sequence set forth in SEQ ID NO: 23.

In a sixth aspect, the invention provides an isolated nucleic acid encoding a G3BP-2 protein fragment of the fourth aspect.

In a seventh aspect, the invention provides an expression vector comprising a nucleic acid of any one of the abovementioned aspects.

In an eighth aspect, the invention relates to use of an antagonist to prevent or disrupt binding between G3BP-2 and another protein.

In one form, the antagonist of the eighth aspect prevents or disrupts binding between a NTF2-like domain of G3BP-2 and said another protein.

In another form, the antagonist is a mimetic of the NTF2-like domain of G3BP-2.

In yet another form, the antagonist binds to the NTF2-like domain.

The antagonist may be a protein.

In one form, the protein comprises an Src homology 3 (SH3) domain.

Preferably, the protein comprises an amino acid sequence as set forth in SEQ ID NO: 6.

The antagonist may be a non-peptide compound.

In a ninth aspect, the invention provides an isolated antigen presenting cell which has been in contact with a G3BP-2 protein, fragment, homolog, variant or derivative thereof, wherein contact includes pulsing or loading the antigen presenting cell with G3BP-2 protein, fragment, homolog, variant or derivative thereof.

In a tenth aspect, the invention provides an isolated antigen presenting cell which has been transfected with a nucleic acid encoding G3BP-2 protein, inclusive of fragments, homologs, variants and derivatives thereof.

The isolated antigen presenting cell of the ninth and tenth aspects is preferably a dendritic cell.

The G3BP-2 protein, inclusive of a fragment, a homolog, a variant and a derivative thereof of the ninth and tenth aspects preferably comprises an amino acid sequence as set forth in SEQ ID NO: 5.

The G3BP-2 fragment of the ninth and tenth aspects preferably comprises an amino acid sequence selected from the group consisting of: KLPNFGFVV [SEQ ID NO: 1] and IMFRGEVRL [SEQ ID NO: 2].

In an eleventh aspect, the invention provides an isolated lymphocyte cell that is G3BP-2 antigen specific.

Preferably, the isolated lymphocyte cell is a cytotoxic T-lymphocyte.

Preferably, the lymphocyte cell is G3BP-2 antigen specific for a protein, inclusive of fragments, homologs, variants and derivatives thereof, comprising an amino acid sequence as set forth in SEQ ID NO: 5.

Preferably, the G3BP-2 protein fragment comprises an amino acid sequence as set forth in SEQ ID NO: 1 or SEQ ID NO: 2.

In a twelfth aspect, the invention provides a pharmaceutical composition comprising at least one active, wherein the active is selected from the group consisting of: a protein, a nucleic acid or an isolated cell of any one of the above aspects.

In a thirteenth aspect, the invention provides a method for preventing or treating breast cancer in a mammal including the step of administering to said mammal a pharmaceutical composition comprising at least one active, wherein the active is selected from the group consisting of: a protein, a nucleic acid, a mimetic of the NTF2-like domain of G3BP-2, an antagonist that prevents or disrupts binding between a NTF2-like domain of G3BP-2 and another protein or isolated cell of any one of the above-mentioned aspects.

Preferably the mammal is human.

In a fourteenth aspect, the invention provides a method for modulating cell proliferation including the step of administering to an animal or isolated cell, an active which prevents or disrupts binding between G3BP-2 and another protein.

Preferably the animal is human.

In a fifteenth aspect, the invention provides a method for isolating a molecule that binds G3BP-2, including the step of determining if one or more candidates in a sample bind to the NTF2-like domain of G3BP-2.

In one form, the molecule is an antagonist.

The antagonist may be a protein or a non-protein molecule.

In a sixteenth aspect, the invention provides a method for diagnosing breast cancer in a mammal including the steps of comparing G3BP-2 protein expression in a test sample obtained from the mammal with G3BP-2 in a reference sample, wherein if the expression of G3BP-2 in the test sample is different than the reference sample, the mammal is diagnosed with an increased likelihood of having breast cancer.

G3BP-2 protein expression may be detected using an antibody.

The antibody may bind to a G3BP-2 protein, inclusive of a fragment, a homolog, a variant and a derivative thereof, comprising an amino acid sequence as set forth in SEQ ID NO: 5.

In one form, the antibody binds to a G3BP-2 protein fragment comprising an amino acid sequence SATPPPAEPASLPQEPPKPRV [SEQ ID NO: 3].

The G3BP-2 fragment may comprise a NTF2-like domain.

Preferably, the mammal is human.

In a seventeenth aspect, the invention provides a method for diagnosing breast cancer in a mammal including the step of detecting a G3BP-2 nucleic acid or fragment thereof in a test sample obtained from the mammal.

Preferably, the test sample is breast tissue.

Preferably, the mammal is human.

In an eighteenth aspect, the invention provides a method of immunising a mammal against breast cancer, including the step of administering to said mammal an immunogenic agent comprising at least one active selected from the group consisting of:
(1) a G3BP-2 protein;
(2) a fragment, a homolog, a variant or a derivative of (1);
(3) a G3BP-2 nucleic acid;
(4) a fragment, a homolog, a variant or a derivative of (3);
(5) an isolated antigen presenting cell that has been contacted with (1) or (2); and
(6) an isolated antigen presenting cell that has been transfected with a nucleic acid of (3) or (4).

The immunisation may be preventative or as a treatment for an animal with breast cancer.

The G3BP-2 protein fragment is preferably selected from the group consisting of: KLPNFGFVV [SEQ ID NO: 1] and IMFRGEVRL [SEQ ID NO: 2].

The antigen presenting cell is preferably a dendritic cell.

Preferably, the mammal is human.

The isolated protein and nucleic acid, and methods according to the aforementioned aspects of the invention are useful in therapeutic or prophylactic treatments of breast cancer and diagnosis thereof. As will be described in more detail hereinafter, disruption of interactions between G3BP-2 and another protein or endogenous binding partner may inhibit tumour proliferation.

### BRIEF DESCRIPTION OF THE FIGURES AND TABLES

FIG. 1 is a nucleotide sequence of HsaG3BP-2a [SEQ ID NO: 4] and encoded amino acid sequence [SEQ ID NO: 5]; and

FIG. 2 is an amino acid sequence [SEQ ID NO: 6] of the N-terminus of rasGAP¹²⁰, which includes a SH3 domain;

FIG. 3 shows a clustal alignment of the G3BP family of proteins, mouse MmuG3BP2a [SEQ ID NO:7], human HsaG3BP2a [SEQ ID NO:5], mouse MmuG3BP2b [SEQ ID NO:8], human HsaG3PB2b [SEQ ID NO:9], human HsaG3BP1 [SEQ ID NO:10] and mouse MmuG3BP1 [SEQ ID NO:11];

FIG. 4 shows analysis of protein-protein interactions between the SH3 domain of N-terminal rasGAP¹²⁰ and G3BP;

FIG. 5 shows Western blot analysis of G3BP expression in different tissues;

FIGS. 6A and 6B shows immunohistochemistry of adult mouse tissues probed with anti-G3BP-1 and anti-G3BP-2 antibodies;

FIGS. 7A and 7B show G3BP-2 immunohistochemistry of two human breast cancers. Panel A is an in situ ductal tumour and panel B shows an infiltrating cancer;

FIG. 8A-T shows immunohistochemical staining of breast tumour sections and immunofluorescence of synchronised NIH 3T3 cells with antibodies specific for G3BPs. Panels A-C are breast tumour sections stained using an antibody specific for G3BP-1. Panels A and B represent stained IDC while. Panel C shows a small section of stained normal ducts (ND). Panels D-O show immunohistochemistry of G3BP-2 in human breast tumours. Panel D shows a normal lobe, Panels E and F show normal ducts cut transverse and longitudinally, respectively (CT denotes connective tissue). Panels G and H show a normal duct adjacent to an IDC (DC denotes ductal carcinoma). Panel I shows an IDC which does not express G3BP-2. Panel J shows an IDC adjacent to normal connective tissue. The arrow indicates cells within the connective tissue which stain positive for G3BP-2 in the nucleus. Panel K shows a lower magnification of an IDC (left side) adjacent to normal connective tissue. Panels L-O illustrate a variety of G3BP-2 subcellular localisations in human breast cancer. All panels are ductal carcinomas from different patients. Panel L shows cytoplasmic localisation of G3BP-2. Panels M and N show nuclear localisation of G3BP-2 in two different cases of breast cancer; cytoplasmic expression is also observed in these sections. Panel O shows G3BP-2 expression around the nuclear envelope region; cytoplasmic staining is also observed. Panels P-T show the immunofluorescence of synchronised NIH 3T3 cells. Panel P shows the sub-cellular localisation of G3BP-2 in cells in G0 phase (time=0). The time after serum stimulation and hence cell cycle commencement is 2 hours, 5 hours, 9 hours and 12 hours for Panels Q, R, S and T, respectively.

FIG. 9 illustrates antibody specificity of G3BP-2 antibodies. The breast cancer cell line MDA-MB-435 and the cervical cancer cell line HeLa (labeled 435 and HeLa respectively) were lysed and equal amounts of protein were resolved by SDS-PAGE. The samples were transferred to a membrane and probed with either the polyclonal G3BP-2 antibody or the commercial G3BP-1 antibody as indicated (Panel A). Purified recombinant G2BP-2b (lane 1), G3BP-1 (lane 2) and G3BP-2a (lane 3) were resolved by SDS-PAGE along with four different truncations of G3BP-2a, N1 (lane 4), C1 (lane 5), C2 (lane 6), N2 (lane 7). These samples were transferred to a membrane and probed with the G3BP-2 antibody (Panel B). Panel C is a schematic representation of G3BP-2a and the recombinant G3BP-2a truncations (N1, N2, C1, C2). It also illustrates the region in which the G3BP-2 polyclonal antibody binds. Inset shows the sub-domains within G3BP-2a.;

FIG. 10 shows G3BP-2 peptide binding to HLA-A*0201 measured by T2 binding assay; and

FIG. 11 is a graph showing data for a chromium release cytotoxic assay using different effector cell: target cell ratios.

Table 1: shows exemplary conservative substitutions in the polypeptide; and
Table 2 summarises expression of G3BP-2 in 58 breast tumour sections. DCIS=ductal carcinoma in situ, IDC=infiltrating ductal carcinoma, LCIS=lobular carcinoma in situ, ILC=infiltrating lobular carcinoma. Grade is assigned according to a range of factors—a well-differentiated tumour is generally assigned a grade 1 while a poorly differentiated tumour is assigned a grade 3. ER=oestrogen receptor status. Node status refers to the presence (+) or absence (−) of tumour in the lymph nodes. NG=not graded. ND=not determined. The column labelled cytoplasm indicates the level of expression of G3BP-2 in the cytoplasm of cells (1+=low, 2+=medium, 3+=high). Unless stated otherwise, staining was present in greater than 75% of cell population. The column labelled nucleus indicates the presence (+) or absence (−) of G3BP-2 in the nucleus of cancer cells.

Table 3 summarises of G3BP-1 in 24 breast tumour sections. DCIS=ductal carcinoma in situ, IDC=infiltrating ductal carcinoma, LCIS=lobular carcinoma in situ, ILC=infiltrating lobular carcinoma. Grade is assigned according to a range of factors—a well-differentiated tumour is generally assigned a grade 1 while a poorly differentiated tumour is assigned a grade 3. ER=oestrogen receptor status. Node status refers to the presence (+) or absence (−) of tumour in the lymph nodes. NG=not graded. ND=not determined. The column labelled cytoplasm indicates the level of expression of G3BP-1 in the cytoplasm of cells (1+=low, 2+=medium, 3+=high). Unless stated otherwise, staining was present in greater than 75% of cell population. The column labelled nucleus indicates the presence (+) or absence (−) of G3BP-1 in the nucleus of cells.

### DETAILED DESCRIPTION OF THE INVENTION

Definitions

Unless defined otherwise, all technical and scientific terms used herein have a meaning as commonly understood by those of ordinary skill in the art to which the invention belongs. Although any method and material similar or equivalent to those described herein can be used in the practice or testing of the present invention, preferred methods and materials are described. For the purpose of the present invention, the following terms are defined below.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material includes material in native and recombinant form. For example, G3BP-2 nucleic acids and encoded polypeptides (inclusive of HsaG3BP-2a, HsaG3BP-2b isolated from human; and MmuG3BP-2a and MmuG3BP-2b isolated from mouse) have been respectively isolated from human and mouse.

By “endogenous” nucleic acid or polypeptide is meant a nucleic acid or polypeptide which may be found in a native cell, tissue or animal in isolation or otherwise.

Polypeptide or Protein

By “polypeptide” is also meant “protein”, either term referring to an amino acid polymer, comprising natural and/or non-natural amino acids as are well understood in the art. For example, G3BP-2 may be referred to as both a protein or polypeptide. “Protein” may refer to a peptide, polypeptide, or fragments thereof. “G3BP-2” protein encompasses isoforms thereof, including G3BP-2a and G3BP-2b and all other isoforms, unless a specific isoform is referred to.

A “peptide” is a protein having no more than fifty (50) amino acids.

In one embodiment, a “fragment” includes an amino acid sequence which constitutes less than 100%, but at least 20%, preferably at least 30%, more preferably at least 80% or even more preferably at least 90% of said polypeptide.

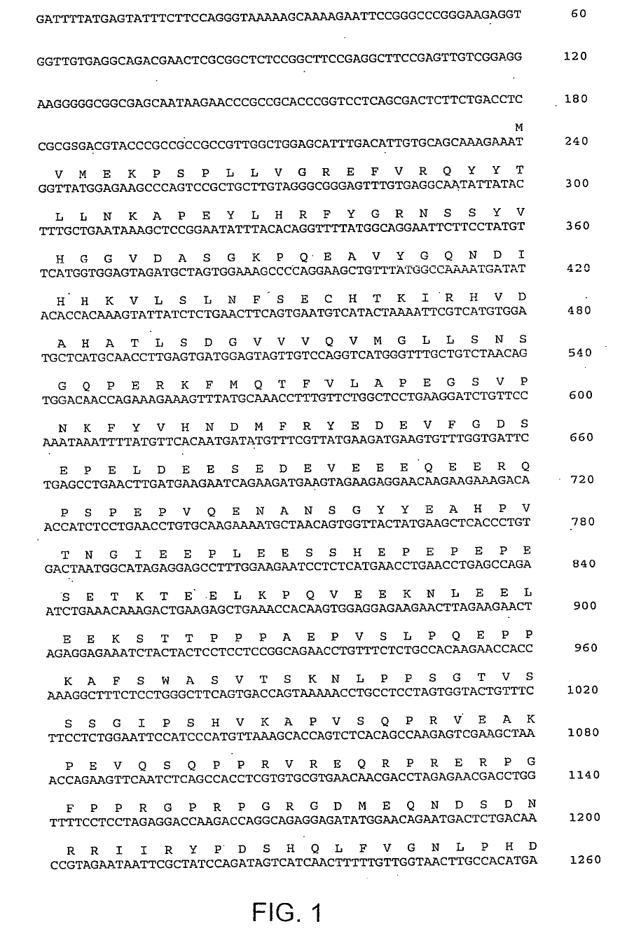

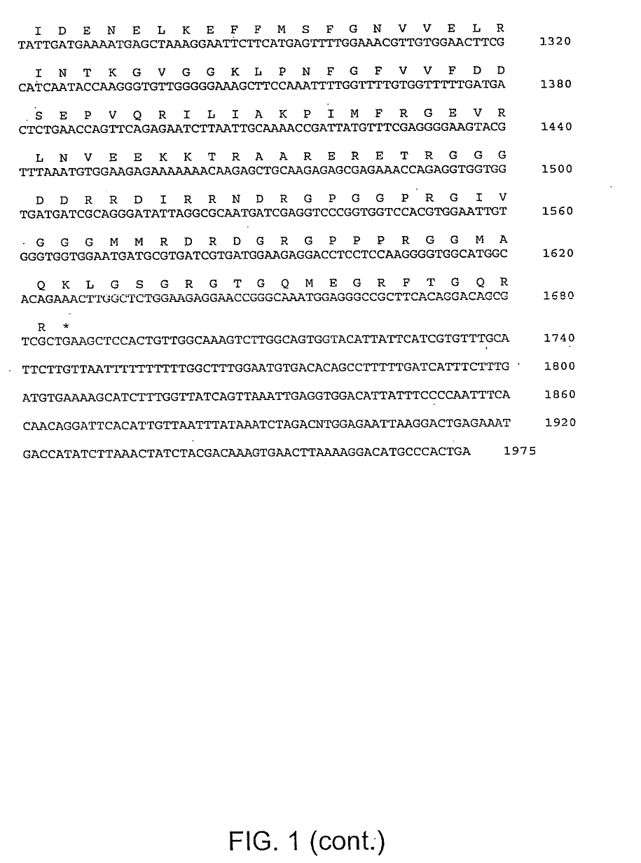

The fragment may also include a “biologically active fragment” which retains biological activity of a given polypeptide or peptide. For example, a biologically active fragment of G3BP-2 comprises a NTF2-like fragment which is associated with binding a SH3 domain. The NTF2-like domain includes amino acid residues 1 to 146 as shown in FIG. 1. It is understood that the fragment may be derived from either a native or a recombinant polypeptide or peptide. The biologically active fragment constitutes at least greater than 1% of the biological activity of the entire polypeptide or peptide, preferably at least greater than 10% biological activity, more preferably at least greater than 25% biological activity and even more preferably at least greater than 50% biological activity.

In another embodiment, a “fragment” is a small peptide, for example of at least 6, preferably at least 10 and more preferably at least 20 amino acids in length, which comprises one or more antigenic determinants or epitopes. Larger fragments comprising more than one peptide are also contemplated, and may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a polypeptide of the invention with a suitable proteinases. The digested fragments can be purified by, for example, high performance liquid chromatographic (HPLC) techniques.

In one form, the invention provides a protein that comprises an NTF2-like domain. The term “comprises” refers to the protein at least having the NTF2-like domain and any additional amino acid sequence.

In another form, the invention provides a protein that consists essentially of the NTF2-like domain. The term “consists essentially of” in relation to a protein refers to a protein that in addition to the stated portion thereof, eg. the NTF2-like domain, consists of no more than 30 additional amino acids located the amino and/or carboxyl terminal end(s) thereof. Preferably, the protein consists of no more than 20 additional amino acids. More preferably, the protein consists of between 1-10 additional amino acids. The additional amino acids or “additions” may comprise a fusion protein, for example those well known in the art including GST and (6×-HIS)-tag as described hereinafter.

In another form, the invention provides a protein that “consist of” the NTF2-like domain. This means a protein comprising an amino acid sequence of only the NTF2-like domain.

The NTF2-like domain is set forth in SEQ ID NO: 5, referring to amino acid residues 1 to 146, wherein amino acid residue 1 is the first methionine (M).

As used herein, “variant” polypeptides are polypeptides of the invention in which one or more amino acids have been replaced by different amino acids. It is well understood in the art that some amino acids may be changed to others with broadly similar properties without changing the nature of the activity of the polypeptide (conservative substitutions). Exemplary conservative substitutions in the polypeptide may be made according to Table 1.

Substantial changes in function are made by selecting substitutions that are less conservative than those shown in Table 1. Other replacements would be non-conservative substitutions and relatively fewer of these may be tolerated. Generally, the substitutions which are likely to produce the greatest changes in a polypeptide's properties are those in which (a) a hydrophilic residue (e.g., Ser or Thr) is substituted for, or by, a hydrophobic residue (e.g. Leu, Ile, Phe or Val); (b) a cysteine or proline is substituted for, or by, any other residue; (c) a residue having an electropositive side chain (e.g., Arg, His or Lys) is substituted for, or by, an electronegative residue (e.g., Glu or Asp) or (d) a residue having a bulky side chain (e.g., Phe or Trp) is substituted for, or by, one having a smaller side chain (e.g., Ala, Ser) or no side chain (e.g., Gly).

Polypeptide and Nucleic Acid Sequence Comparison

Terms used herein to describe sequence relationships between respective nucleic acids and polypeptides include “comparison window”, “sequence identity”, “percentage of sequence identity” and “substantial identity”. Because respective nucleic acids/polypeptides may each comprise (1) only one or more portions of a complete nucleic acid/polypeptide sequence that are shared by the nucleic acids/polypeptides, and (2) one, or more portions which are divergent between the nucleic acids/polypeptides, sequence comparisons are typically performed by comparing sequences over a “comparison window” to identify and compare local regions of sequence similarity. A “comparison window” refers to a conceptual segment of typically at least 6 contiguous residues that is compared to a reference sequence. The comparison window may comprise additions or deletions (i.e., gaps) of about 20% or less as compared to the reference sequence (which does not comprise additions or deletions) for optimal alignment of the respective sequences. Optimal alignment of sequences for aligning a comparison window may be conducted by computerised implementations of algorithms (for example ECLUSTALW and BESTFIT provided by WebAngis GCG, 2D Angis, GCG and GeneDoc programs, incorporated herein by reference) or by inspection and the best alignment (i.e., resulting in the highest percentage homology over the comparison window) generated by any of the various methods selected.

The ECLUSTALW program is used to align multiple sequences. This program calculates a multiple alignment of nucleotide or amino acid sequences according to a method by Thompson, J. D., Higgins, D. G. and Gibson, T. J. (1994). This is part of the original ClustalW distribution, modified for inclusion in EGCG. The BESTFIT program aligns forward and reverse sequences and sequence repeats. This program makes an optimal alignment of a best segment of similarity between two sequences. Optimal alignments are determined by inserting gaps to maximize the number of matches using the local homology algorithm of Smith and Waterman. ECLUSTALW and BESTFIT alignment packages are offered in WebANGIS GCG (The Australian Genomic Information Centre, Building JO3, The University of Sydney, N.S.W 2006, Australia).

Reference also may be made to the BLAST family of programs as for example disclosed by Altschul et al., 1997, Nucl. Acids Res. 25 3389, which is incorporated herein by reference.

A detailed discussion of sequence analysis can be found in Chapter 19.3 of Ausubel et al, supra.

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, U) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, Calif., USA).

As generally used herein, a “homolog” shares a definable nucleotide or amino acid sequence relationship with a nucleic acid or polypeptide of the invention as the case may be.

“Polypeptide homologs” share at least 80%, preferably at least 90% and more preferably at least 95% sequence identity with the amino acid sequences of polypeptides of the invention as hereinbefore described. Polypeptide homologs include, for example G3BP-1. Also included are G3BP-2 isoforms G3BP-2a and G3BP-2b.

Included within the scope of homologs are “orthologs”, which are functionally-related polypeptides and their encoding nucleic acids, isolated from other organisms. For example G3BP-2 polypeptides isolated from human (eg. HsaG3BP-2a, HsaG3BP-2b) and mouse (eg. MmuG3BP-2a, MmuG3BP-2b).

With regard to polypeptide variants, these can be created by mutagenising a polypeptide or by mutagenising an encoding nucleic acid, such as by random mutagenesis or site-directed mutagenesis. Examples of nucleic acid mutagenesis methods are provided in Chapter 9 of CURRENT PROTOCOLS IN MOLECULAR BIOLOGY, Ausubel et al., supra which is incorporated herein by reference.

It will be appreciated by the skilled person that site-directed mutagenesis is best performed where knowledge of the amino acid residues that contribute to biological activity is available. In many cases, this information is not available, or can only be inferred by molecular modeling approximations, for example.

In such cases, random mutagenesis is contemplated. Random mutagenesis methods include chemical modification of proteins by hydroxylamine (Ruan et al., 1997, Gene 188 35), incorporation of dNTP analogs into nucleic acids (Zaccolo et al., 1996, J. Mol. Biol. 255 589) and PCR-based random mutagenesis such as described in Stemmer, 1994, Proc. Natl. Acad. Sci. USA 91 10747 or Shafikhani et al., 1997, Biotechniques 23 304, each of which references is incorporated herein. It is also noted that PCR-based random mutagenesis kits are commercially available, such as the Diversify™ kit (Clontech).

As used herein, “derivative” polypeptides are polypeptides of the invention which have been altered, for example by conjugation or complexing with other chemical moieties or by post-translational modification techniques as would be understood in the art. Such derivatives include amino acid deletions and/or additions to polypeptides of the invention, or variants thereof.

“Additions” of amino acids may include fusion of the peptide or polypeptides or variants thereof with other peptides or polypeptides. Particular examples of such peptides include amino (N) and carboxyl (C) terminal amino acids added for use as “tags”. Use of an N-terminal 6×-His tag for isolating an expressed fusion polypeptide is described herein.

N-terminal and C-terminal tags include known amino acid sequences which bind a specific substrate, or bind known antibodies, preferably monoclonal antibodies. pRSET B vector (ProBond™; Invitrogen Corp.) is an example of a vector comprising an N-terminal 6×-His-tag which binds ProBond™ resin.

Other derivatives contemplated by the invention include, modification to side chains, incorporation of unnatural amino acids and/or their derivatives during peptide or polypeptide synthesis and the use of cross linkers and other methods which impose conformational constraints on the polypeptides, fragments and variants of the invention. Examples of side chain modifications contemplated by the present invention include modifications of amino groups such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with NaBH₄; reductive alkylation by reaction with an aldehyde followed by reduction with NaBH₄; and trinitrobenzylation of amino groups with 2,4,6-trinitrobenzene sulphonic acid (TNBS).

The carboxyl group may be modified by carbodiimide activation via O-acylisourea formation followed by subsequent derivitization, by way of example, to a corresponding amide.

The guanidine group of arginine residues may be modified by formation of heterocyclic condensation products with reagents such as 2,3-butanedione, phenylglyoxal and glyoxal.

Sulphydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulphides with other thiol compounds; reaction with maleimide, maleic anhydride or other substituted maleimide; carboxymethylation with iodoacetic acid or iodoacetamide; and carbamoylation with cyanate at alkaline pH.

Tryptophan residues may be modified, for example, by alkylation of the indole ring with 2-hydroxy-5-nitrobenzyl bromide or sulphonyl halides or by oxidation with N-bromosuccinimide.

Tyrosine residues may be modified by nitration with tetranitromethane to form a 3-nitrotyrosine derivative.

The imidazole ring of a histidine residue may be modified by N-carbethoxylation with diethylpyrocarbonate or by alkylation with iodoacetic acid derivatives.

Examples of incorporating unnatural amino acids and derivatives during peptide synthesis include, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids.

Polypeptides in relation to the invention such as those exemplified in FIG. 1 (inclusive of fragments, variants, derivatives and homologs in general) may be prepared by any suitable procedure known to those of skill in the art.

For example, the polypeptide may be prepared by a procedure including the steps of:
(i) preparing an expression construct which comprises a recombinant nucleic acid of the invention, operably linked to one or more regulatory nucleotide sequences, for example a T7 promoter;
(ii) transfecting or transforming the expression construct into a suitable host cell, for example E. coli; and
(iii) expressing the polypeptide in said host cell.

Preferably, the recombinant nucleic acid of the invention encodes a polypeptide as shown in FIG. 1, or fragment thereof.

Recombinant proteins may be conveniently expressed and purified by a person skilled in the art using commercially available kits, for example “ProBond™ Purification System” available from Invitrogen Corporation, Carlsbad, Calif., USA, herein incorporated by reference. Alternatively, standard molecular biology protocols may be used, as for example described in Sambrook, et al., MOLECULAR CLONING. A Laboratory Manual (Cold Spring Harbor Press, 1989), incorporated herein by reference, in particular Sections 16 and 17; CURRENT PROTOCOLS IN MOLECULAR BIOLOGY Eds. Ausubel et al., (John Wiley & Sons, Inc. 1995-1999), incorporated herein by reference, in particular Chapters 10 and 16; and CURRENT PROTOCOLS IN PROTEIN SCIENCE Eds. Coligan et al., (John Wiley & Sons, Inc. 1995-1999) which is incorporated by reference herein, in particular Chapters 1, 5, 6 and 7.

Nucleic Acids

The term “nucleic acid” as used herein designates single or double stranded mRNA, RNA, cRNA and DNA, said DNA inclusive of cDNA and genomic DNA.

The term “isolated nucleic acid” as used herein refers to a nucleic acid subjected to in vitro manipulation into a form not normally found in nature. Isolated nucleic acid include both native and recombinant (non-native) nucleic acids. For example, a nucleic acid isolated from human or mouse.

A “polynucleotide” is a nucleic acid having eighty (80) or more contiguous nucleotides, while an “oligonucleotide” has less than eighty (80) contiguous nucleotides.

The term “consists essentially of in relation to a nucleic acid refers to a nucleic acid having no more than 90 nucleotides located at the 5′ and/or 3′ thereof. Preferably, the nucleic acid consist of no more than 60 additional nucleic acids, more preferably the nucleic acid consist of between 1-30 nucleotides.

A “probe” may be a single or double-stranded oligonucleotide or polynucleotide, suitably labeled for the purpose of detecting complementary sequences in Northern or Southern blotting, for example.

A “primer” is usually a single-stranded oligonucleotide, preferably having 20-50 contiguous nucleotides, which is capable of annealing to a complementary nucleic acid utemplate” and being extended in a template-dependent fashion by the action of a DNA polymerase such as Taq polymerase, RNA-dependent DNA polymerase or Sequenase™. For example, the following primers were used for chromosomal mapping of human G3BP-1 and G3BP-2.

HsaG3BP-1 Specific Primers:

| | |
|---|---|
| 5′GGAGGCATGGTGCAGAAACCA; | [SEQ ID NO:12] |
| and | |
| 5′CAGGAAAGGGAAGAGAGGGAG | [SEQ ID NO:13] |

HsaG3BP-2 Specific Primers:

| | |
|---|---|
| 5′GTCTTGGCAGTGGTACATTAT; | [SEQ ID NO:14] |
| and | |
| 5′AGTTCACTTTGTCGTAGATAGTTTAAG | [SEQ ID NO:15] |

For the purposes of host cell expression, the recombinant nucleic acid is operably linked to one or more regulatory sequences in an expression vector, for example a T7 promoter.

An “expression vector” may be either a self-replicating extra-chromosomal vector such as a plasmid, or a vector that integrates into a host genome. An example of an expression vector is pRSET B (Invitrogen Corp.) and derivations thereof.

By “operably linked” is meant that said regulatory nucleotide sequence(s) is/are positioned relative to the recombinant nucleic acid of the invention to initiate, regulate or otherwise control transcription.

Regulatory nucleotide sequences will generally be appropriate for the host cell used for expression. Numerous types of appropriate expression vectors and suitable regulatory sequences are known in the art for a variety of host cells.

Typically, said one or more regulatory nucleotide sequences may include, promoter sequences, leader or signal sequences, ribosomal binding sites, transcriptional start and termination sequences, translational start and termination sequences, and enhancer or activator sequences.

Constitutive or inducible promoters as known in the art are contemplated by the invention. The promoters may be either naturally occurring promoters, or hybrid promoters that combine elements of more than one promoter. For example, the lac promoter is inducible by IPTG.

The expression vector may further comprise a selectable marker gene to allow the selection of transformed host cells. Selectable marker genes are well known in the art and will vary with the host cell used. For example, an ampicillin resistance gene for selection of positively transformed host cells when grown in a medium comprising ampicillin.

The expression vector may also include a fusion partner (typically provided by the expression vector) so that the recombinant polypeptide of the invention is expressed as a fusion polypeptide with the fusion partner. An advantage of fusion partners is that they assist identification and/or purification of the fusion polypeptide. Identification and/or purification may include using a monoclonal antibody or substrate specific for the fusion partner, for example a 6×-His tag or GST. A fusion partner may also comprise a leader sequence for directing secretion of a recombinant polypeptide, for example an alpha-factor leader sequence.

Well known examples of fusion partners include hexahistidine (6×-HIS)-tag, N-Flag, Fc portion of human IgG, glutathione-5-transferase (GST) and maltose binding protein (MBP), which are particularly useful for isolation of the fusion polypeptide by affinity chromatography. For the purposes of fusion polypeptide purification by affinity chromatography, relevant matrices for affinity chromatography may include nickel-conjugated or cobalt-conjugated resins, fusion polypeptide specific antibodies, qlutathione-conjugated resins, and amylose-conjugated resins respectively. Some matrices are available in “kit” form, such as the ProBond™ Purification System (Invitrogene Corp.) which incorporates a 6×-His fusion vector and purification using ProBond™ resin.

In order to express the fusion polypeptide, it is necessary to ligate a nucleic acid according to the invention into the expression vector so that the translational reading frames of the fusion partner and the nucleotide sequence of the invention coincide.

The fusion partners may also have protease cleavage sites, for example enterokinase (available from Invitrogen Corp. as EnterokinaseMax™), Factor Xₐ or Thrombin, which allow the relevant protease to digest the fusion polypeptide of the invention and thereby liberate the recombinant polypeptide of the invention therefrom. The liberated polypeptide can then be isolated from the fusion partner by subsequent chromatographic separation.

Fusion partners may also include within their scope “epitope tags”, which are usually short peptide sequences for which a specific antibody is available.

As hereinbefore, polypeptides of the invention may be produced by culturing a host cell transformed with an expression construct comprising a nucleic acid encoding a polypeptide, or polypeptide homolog, of the invention. The conditions appropriate for polypeptide expression will vary with the choice of expression vector and the host cell. For example, a nucleotide sequence of the invention may be modified for successful or improved polypeptide expression in a given host cell. Modifications include altering nucleotides depending on preferred codon usage of the host cell. Alternatively, or in addition, a nucleotide sequence of the invention may be modified to accommodate host specific splice sites or lack thereof. These modifications may be ascertained by one skilled in the art.

Host cells for expression may be prokaryotic or eukaryotic.

Useful prokaryotic host cells are bacteria.

A typical bacteria host cell is a strain of E. coli.

Useful eukaryotic cells are yeast, SF9 cells that may be used with a baculovirus expression system as described herein, and other mammalian cells.

The recombinant polypeptide may be conveniently prepared by a person skilled in the art using standard protocols as for example described in Sambrook, et al., MOLECULAR CLONING. A Laboratory Manual (Cold Spring Harbor Press, 1989), incorporated herein by reference, in particular Sections 16 and 17; CURRENT PROTOCOLS IN MOLECULAR BIOLOGY Eds. Ausubel et al., (John Wiley & Sons, Inc. 1995-1999), incorporated herein by reference, in particular Chapters 10 and 16; and CURRENT PROTOCOLS IN PROTEIN SCIENCE Eds. Coligan et al., (John Wiley & Sons, Inc. 1995-1999) which is incorporated by reference herein, in particular Chapters 1, 5 and 6.

In one embodiment, nucleic acid homologs encode polypeptide homologs of the invention, inclusive of variants, fragments and derivatives thereof.

In another embodiment, nucleic acid homologs share at least 60%, preferably at least 70%, more preferably at least 80%, and even more preferably at least 90% sequence identity with the nucleic acids of the invention.

In yet another embodiment, nucleic acid homologs hybridise to nucleic acids of the invention under at least low stringency conditions, preferably under at least medium stringency conditions and more preferably under high stringency conditions.

“Hybridise and Hybridisation” is used herein to denote the pairing of at least partly complementary nucleotide sequences to produce a DNA-DNA, RNA-RNA or DNA-RNA hybrid. Hybrid sequences comprising complementary nucleotide sequences occur through base-pairing.

In DNA, complementary bases are:
(i) A and T; and
(ii) C and G.

In RNA, complementary bases are:
(i) A and U; and
(ii) C and G.

In RNA-DNA hybrids, complementary bases are:
(i) A and U;
(ii) A and T; and
(iii) G and C.

Modified purines (for example, inosine, methylinosine and methyladenosine) and modified pyrimidines (thiouridine and methylcytosine) may also engage in base pairing.

“Stringency” as used herein, refers to temperature and ionic strength conditions, and presence or absence of certain organic solvents and/or detergents during hybridisation. The higher the stringency, the higher will be the required level of complementarity between hybridizing nucleotide sequences.

“Stringent conditions” designates those conditions under which only nucleic acid having a high frequency of complementary bases will hybridize.

Reference herein to low stringency conditions includes and encompasses:—
(i) from at least about 1% v/v to at least about 15% v/v formamide and from at least about 1 M to at least about 2 M salt for hybridisation at 42° C., and at least about 1 M to at least about 2 M salt for washing at 42° C.; and
(ii) 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature.

Medium stringency conditions include and encompass:—
(i) from at least about 16% v/v to at least about 30% V/v formamide and from at least about 0.5 M to at least about 0.9 M salt for hybridisation at 42° C., and at least about 0.5 M to at least about 0.9 M salt for washing at 42° C.; and
(ii) 1% Bovine Serum Albumin (BSA), 1 mM EDTA, 0.5 M NaHPO₄ (pH 7.2), 7% SDS for hybridization at 65° C. and (a) 2×SSC, 0.1% SDS; or (b) 0.5% BSA, 1 mM EDTA, 40 mM NaHPO₄ (pH 7.2), 5% SDS for washing at 42° C.

High stringency conditions include and encompass:—
(i) from at least about 31% v/v to at least about 50% v/v formamide and from at least about 0.01 M to at least about 0.15 M salt for hybridisation at 42° C., and at least about 0.01 M to at least about 0.15 M salt for washing at 42° C.;
(ii) 1% BSA, 1 mM EDTA, 0.5 M NaHPO₄ (pH 7.2), 7% SDS for hybridization at 65° C., and (a) 0.1×SSC, 0.1% SDS; or (b) 0.5% BSA, 1 mM EDTA, 40 mM NaHPO₄ (pH 7.2), 1% SDS for washing at a temperature in excess of 65° C. for about one hour; and
(iii) 0.2×SSC, 0.1% SDS for washing at or above 68° C. for about 20 minutes.

In general, the Tₘ of a duplex DNA decreases by about 1° C. with every increase of 1% in the number of mismatched bases.

Notwithstanding the above, stringent conditions are well known in the art, such as described in Chapters 2.9 and 2.10 of Ausubel et al., supra, which are herein incorporated by reference. A skilled addressee will also recognize that various factors can be manipulated to optimize the specificity of the hybridization. Optimization of the stringency of the final washes can serve to ensure a high degree of hybridization.

Typically, complementary nucleotide sequences are identified by blotting techniques that include a step whereby nucleotides are immobilized on a matrix (preferably a synthetic membrane such as nitrocellulose), a hybridization step, and a detection step. Southern blotting is used to identify a complementary DNA sequence; Northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al., supra, at pages 2.9.1 through 2.9.20, herein incorporated by reference.

According to such methods, Southern blotting involves separating DNA molecules according to size by gel electrophoresis, transferring the size-separated DNA to a synthetic membrane, and hybridizing the membrane bound DNA to a complementary nucleotide sequence.

In dot blotting and slot blotting, DNA samples are directly applied to a synthetic membrane prior to hybridization as above.

An alternative blotting step is used when identifying complementary nucleic acids in a cDNA or genomic DNA library, such as through the process of plaque or colony hybridisation. Other typical examples of this procedure are described in Chapters 8-12 of Sambrook et al., supra which are herein incorporated by reference.

Typically, the following general procedure can be used to determine hybridisation conditions. Nucleic acids are blotted/transferred to a synthetic membrane, as described above. A nucleotide sequence of the invention is labeled as described above, and the ability of this labeled nucleic acid to hybridise with an immobilized nucleotide sequence analysed.

A skilled addressee will recognise that a number of factors influence hybridisation. The specific activity of radioactively labeled polynucleotide sequence should typically be greater than or equal to about 10⁸ dpm/μg to provide a detectable signal. A radiolabeled nucleotide sequence of specific activity 10⁸ to 10⁹ dpm/μg can detect approximately 0.5 pg of DNA. It is well known in the art that sufficient DNA must be immobilised on the membrane to permit detection. It is desirable to have excess immobilised DNA, usually 10 pg. Adding an inert polymer such as 10% (w/v) dextran sulfate (MW 500,000) or polyethylene glycol 6000 during hybridisation can also increase the sensitivity of hybridisation (see Ausubel et al., supra at 2.10.10).

To achieve meaningful results from hybridisation between a nucleic acid immobilised on a membrane and a labeled nucleic acid, a sufficient amount of the labeled nucleic acid must be hybridised to the immobilised nucleic acid following washing. Washing ensures that the labeled nucleic acid is hybridised only to the immobilised nucleic acid with a desired degree of complementarity to the labeled nucleic acid.

Methods for detecting labeled nucleic acids hybridised to an immobilised nucleic acid are well known to practitioners in the art. Such methods include autoradiography, chemiluminescent, fluorescent and colourimetric detection.

Nucleic acid homologs of the invention may be prepared according to the following procedure:
(i) obtaining a nucleic acid extract from a suitable host, for example a bacterial species;
(ii) creating primers which are optionally degenerate wherein each comprises a portion of a nucleotide sequence of the invention; and
(iii) using said primers to amplify, via nucleic acid amplification techniques, one or more amplification products from said nucleic acid extract.

As used herein, an “amplification product” refers to a nucleic acid product generated by nucleic acid amplification techniques.

Suitable nucleic acid amplification techniques are well known to the skilled addressee, and include PCR as for example described in Chapter 15 of Ausubel et al. supra, which is incorporated herein by reference; strand displacement amplification (SDA) as for example described in U.S. Pat. No. 5,422,252 which is incorporated herein by reference; rolling circle replication (RCR) as for example described in Liu et al., 1996, J. Am. Chem. Soc. 118 1587 and International application WO 92/01813; and Lizardi and Caplan, International Application WO 97/19193, which are incorporated herein by reference; nucleic acid sequence-based amplification (NASBA) as for example described by Sooknanan et al., 1994, Biotechniques 17 1077, which is incorporated herein by reference; ligase chain reaction (LCR) as for example described in International Application WO89/09385 which is incorporated herein by reference; and Q-β replicase amplification as for example described by Tyagi et al., 1996, Proc. Natl. Acad. Sci. USA 93 5395 which is incorporated herein by reference. Preferably, amplification is by PCR using primers disclosed herein.

G3BP-2 and Breast Cancer

G3BP-2 protein was found to be in breast cancers that are derived from epithelial tissues and non-proliferative tissues. This was an unexpected result, at least partly because it was not known that expression would be shown to be specific to tumours and that up-regulation of G3BP-2 would occur so early in the development of the tumour. G3BP-2 is up-regulated in approximately 80% of breast cancers studied. This compares to genes that are well recognised as causing breast cancers such as Brca1, which is found to be mutated in 15% of familial breast cancers (percentages depend on the country of the study). Familial breast cancers only represent 15-30% of total breast cancers, which means that Brca1 is causative of only 2-5% of all breast cancers.

The inventors have proposed a model that suggests G3BP-2 migrates to the nucleus of the cell to pick up transcripts and export them to the ribosome so that translation of selected gene transcripts can be regulated at the level of the ribosome. The inventors have proposed this model because of a surprising finding that G3BP-2 can be immunoprecipitated with ribosomal proteins normally associated with polysomes. This suggests that a possible method of action may be to export the transcripts of oncogenes that regulate cell cycle to the ribosome to up-regulate their transcription and thereby enhancing cancer progression. Interestingly it also suggests that G3BP-2 may represent a connection between signal transduction and RNA-processing.

G3BP-2 may not actually be causative of breast cancer, but may be required by the cancer to cause proliferation and thereby account for its up-regulation in 80% of breast cancers. G3BP-2 is specifically localised to sub-cellular compartments in a cell cycle dependent manner, moving into the nucleus during proliferation and then back out to the cytoplasm. G3BP-2 appears to “short-circuit” normal ras-GAP¹²⁰ signalling by receiving messages directly from GAP¹²⁰ and moving into the cell nucleus. In the nucleus G3BP-2 is most likely binding with transcript targets (eg. c-myc) and exporting them from the nucleus to the ribosomes in the cytoplasm. The inventors have co-immunoprecipitated G3BP-2 with a series of polypeptides normally associated with translational active ribosomes. Accordingly, G3BP-2 may be facilitating increased proliferation of breast cancers by allowing the up-regulation of oncogenic gene transcripts (eg. c-myc). Therapeutics designed to block the activity of G3BP-2, in particular at the N-terminal NTF2-like domain, may limit cancer progression.

Cloning, Sequence Homology and Structural Homology

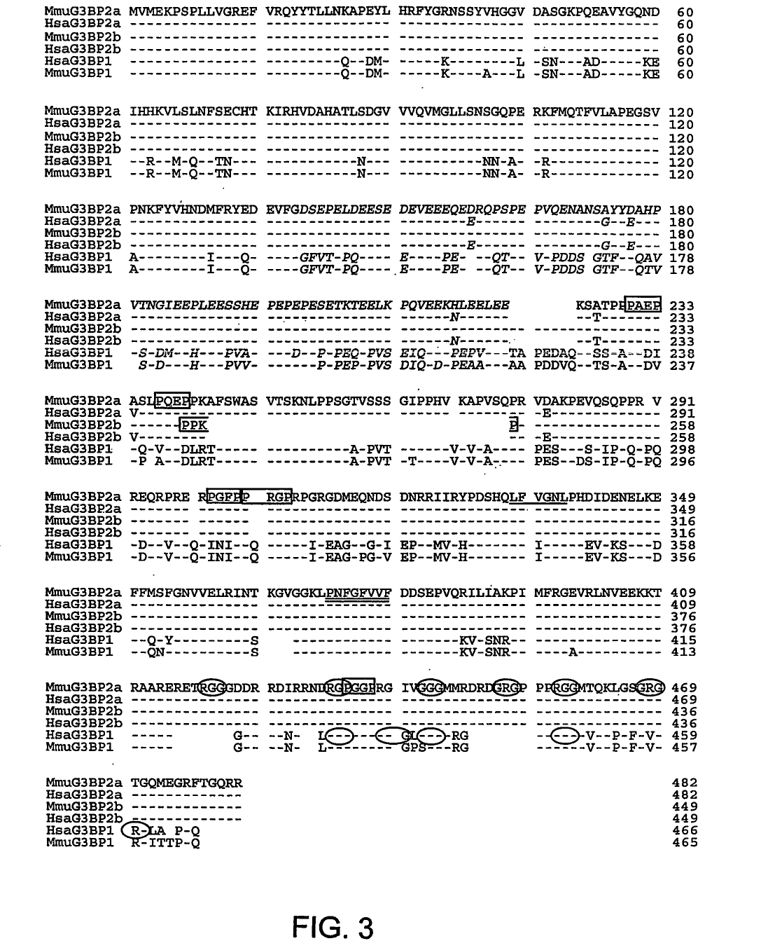

The inventors previously reported cloning MmuG3BP-2 (MMU65313) in a general PCR-based screening for RRM-containing proteins (Kennedy et al. 1997). This was achieved by using degenerate primers designed to consensus sequences within the RRM (Birney et al., 1993) and using the amplified PCR product to screen a late-primitive-streak stage mouse embryo cDNA library to isolate a full-length cDNA. Due to the conserved sequence homology between the G3BP genes, the coding region of the MmuG3BP-2 cDNA can be used as a useful tool to recognise both human G3BP-2 and MmuG3BP-1 in Northern analysis and library screening. The inventors used the coding region of MmuG3BP-2 to isolate and clone MmuG3BP-1 (MMAB1927) and human G3BP-2 (HsaG3BP-2) from the late-primitive-streak stage mouse embryo cDNA library and a foetal human brain cDNA library respectively. The clones were sequenced and analysed to identify Met start codons, open reading frames and stop codons. Protein sequence comparison (FIG. 3) between HsaG3BP-2 and MmuG3BP-2 show 98.5% identity, HsaG3BP-2 and HsaG3BP-1 (HS3251910) show 65% identity and HsaG3BP-1 shares 94.4% sequence identity with MmuG3BP-1. In FIG. 3, amino acids are shown in single letter format and grouped in blocks of 10. Numbering at the end of the line indicates the amino acid position within the indicated protein. Dashes within the aligned sequences indicate conserved amino acids with respect to HsaG3BP-2a, amino acid changes between proteins have been indicated by the appropriate substitution. Spaces within the aligned proteins indicate gaps inserted into the sequences to maintain co-linearity. Boxes represent proline rich sequences (PxxP). Sequences in italics indicate the acid-rich domain. Ovals represent components of an RGG domain, note that the RGG domains of G3BP-1 and G3BP-2 are divergent. Underlined and double underlined sequences indicate RNP-2 and RNP-1 respectively.

Screening of the libraries also revealed that at least two different G3BP-2 isoforms are produced in both mouse and human. The alternative splicing event, which is 100% conserved between mouse and human, deletes 99 nucleotides from the coding region and does not introduce any stop codons nor a frame shift. Both these transcripts are translated in vivo as was confirmed by Western blot analysis and recombinant protein expression (see below). The longer isoform has been designated G3BP-2a (human G3BP-2a accession number AF145285, mouse G3BP-2a accession number AF145285). The shorter protein isoform is referred to as G3BP-2b (human G3BP-2b accession number AF053535 and AF051311 and mouse G3BP-2b accession number MMU65313) differs from G3BP-2a by a 33 amino acid deletion from the central region of the primary structure (FIG. 3). A third MmuG3BP-2 transcript was detected and cloned (referred to herein as G3BP-2c); however, sequence analysis indicated that translation of this transcript would lead to a truncated gene product and so far no corresponding protein has been detected in cells or tissues suggesting that it may not be translated.

All G3BPs share highly conserved RNP-1 and RNP-2 sequences, which are consensus motifs of RRMs. The most notable difference between G3BP-2 and G3BP-1 RRMs is a Val to Ile substitution in the RNP-2 consensus sequence (FIG. 3). The structure of the G3BP RRM has been reported elsewhere (Kennedy et al. 1997). In addition, the G3BPs contain a conserved acid-rich domain and an RGG domain (Birney et al. 1993), both of which are commonly found in RNA-binding proteins. It should be noted that there are considerable differences in the RGG domain structure between G3BP-1 and G3BP-2 (see FIG. 3) and this may result in a different RNA target specificity. Although acid-rich domains are found in association with a variety of RNA-binding proteins such as hnRNP C and nucleolin, the function of this domain remains unclear and maybe involved in protein-protein interactions. The most significant difference between the G3BP-1 and G3BP-2 proteins lies in the number of potential SH3 domain-binding motifs (PxxP, where x represents any amino acid) (Lee et al. 1996). The G3BP-2a protein comprises a cluster of four PxxP motifs between the acid-rich and RRM domains whereas G3BP-2b contains five in the homologous region (FIG. 3). The additional proline-rich PxxP motif in G3BP-2b is generated by the 33 amino acids spliced out of G3BP-2a. In contrast to the multiple PxxP clusters found in G3BP-2s, G3BP-1 contains only one such motif in the homologous region of the protein (FIG. 3). Furthermore, both G3BP-1 and G3BP-2 comprise a conserved PxxP motif (PGGP) within their non-conserved RGG domains (FIG. 3). The specific conservation of the minimal SH3 domain-binding motif within a region of the protein, which is generally not conserved, may suggest a retained function although this remains to be determined. The overall differences in the number of potential SH3 domain-binding motifs between the G3BPs may indicate that in vivo they may bind different SH3 domain-containing partners or have different affinities for the same protein.

Protein Expression in Insect Cells

MmuG3BP-2a and 2b cDNAs encode proteins whose predicted sizes are 58.2 kDa and 52 kDa respectively. The expressed recombinant proteins have apparent molecular weights of 68.5 kDa and 62 kDa respectively, as determined by SDS-PAGE. These differences in predicted and apparent masses are consistent with an increase in mass due to post translational modifications and are similar to those reported for HsaG3BP-1 (predicted mass of 52, observed apparent mass of 68 kDa) (Parker et al. 1996).

Mapping of G3BP-rasGAP¹²⁰ Interactions

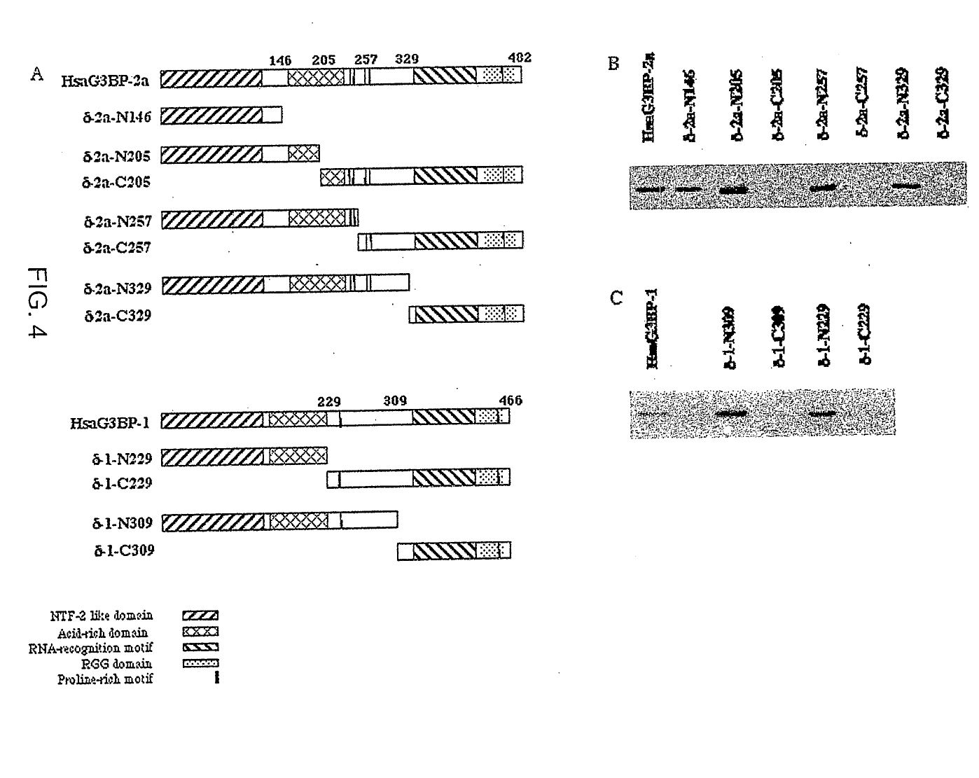

Interactions between G3BP-1 and rasGAP¹²⁰ have been reported and show that G3BP-1 specifically interacts with the SH3 domain of rasGAP¹²⁰ (Parker et al. 1996) implicating G3BP in the rasGAP¹²⁰ signal transduction pathway (see also Pazman et al. 2000). However, the region within the G3BPs responsible for the observed interaction with rasGAP¹²⁰ has not been thoroughly investigated. Initially it was presumed that the interactions would be facilitated through proline rich motifs that are known to interact with SH3 domains (Lee et al. 1996). To further map the interactions between G3BPs and the SH3 domain of rasGAP¹²⁰ several GST-G3BP peptide fusions were expressed (FIG. 4) and probed in bead binding assays with His-tagged N-terminal rasGAP¹²⁰ peptides. The G3BP peptide constructs were designed to represent truncated proteins containing single or multiple domains as well as peptides that would contain isolated proline rich motifs (FIG. 4). In the assays GST-G3BP peptides are bound to glutathione beads and His-tagged N-terminal rasGAP¹²⁰ is added to the different constructs. Specific protein-protein interactions between the G3BP peptides and the SH3 domain of rasGAP¹²⁰ are detected by running the bound proteins on a Western blot and identifying His-tagged rasGAP¹²⁰ by probing with an anti-His antibody. The results show (FIG. 4) that the interaction between the SH3 domain of rasGAP¹²⁰ maps to the NTF2-like domain contained within the N-terminal domain of the G3BPs but not the proline rich motifs. No interactions were detected with the other domains of G3BP including the Acid-Rich domain, the RRM or the RGG-rich domain (FIG. 4).

FIG. 4A shows a schematic representation of sub-domains and motifs contained within G3BP-2a and G3BP-1 proteins and includes the N-terminal NTF2-like, Acid-rich, RGG and proline-rich domains as well as the RNA-recognition motif (see insert for details). Below the respective full length proteins are shown various truncated peptides that were expressed as GST-fusion proteins to map interactions with the N-terminal SH3 domain containing region of rasGAP²⁰. The numbering corresponds to the amino acid at the site of the peptide truncation (ie. δ-2a-N146 represents the truncated G3BP-2a peptide including the N-terminal amino acids 1 to 146), the relative position of these truncations is shown to approximate scale in the full length proteins. FIGS. 4B and 4C show Western blot analysis of Glutathione beads bound with various GST-G3BP peptides and probed with the His-tagged N-terminal SH3 domain containing region of rasGAP¹²⁰.

G3BP-rasGAP¹²⁰ interactions were determined by probing the Western blots with anti-His antibodies. The results show that the N-terminal SH3 domain containing region of rasGAP¹²⁰ interacted with the NTF2 like domain of G3BP-2a (δ-2a-N146, panel B) and any peptide of G3BP-2a that comprised the NTF2 like domain (δ-2a-N205, δ-2a-N257, δ-2a-N329 and full length G3BP-2a, panel B). The results obtained from G3BP-1 are consistent with these results from G3BP-2a and show that full length G3BP-1, δ-1-N229 and δ-1-N309 interact with the N-terminal SH3 domain containing region of rasGAP¹²⁰. Truncated peptides of either G3BP-2a or G3BP-1 that did not contain an intact NTF2-like domain failed to bind with the SH3 region of rasGAP¹²⁰.

The results reported herein were confirmed using far-Western protocols (data not shown) and are consistent with the data obtained from the bead binding assays.

G3BPs have a Tissue Specific Expression

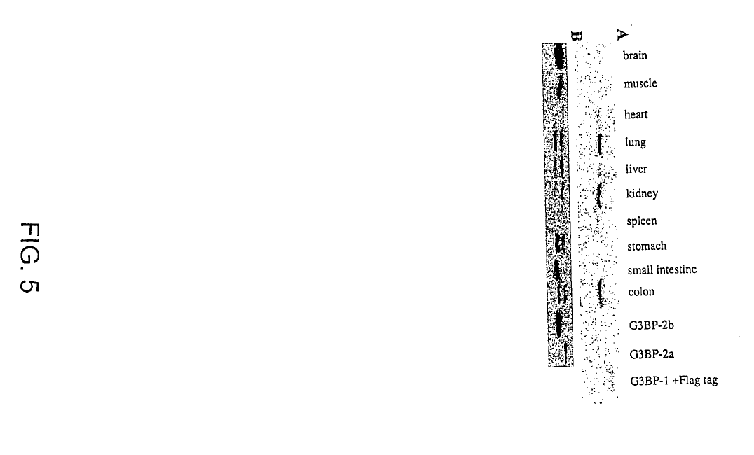

Antibodies raised against isoform specific synthetic peptides determined from G3BP-1 and G3BP-2 sequences were used to probe western blots of total cell lysates from adult mouse tissues (FIG. 5). FIG. 5, panel A shows tissues probed with anti-G3BP-1 polyclonal antibodies whereas panel B shows a collage of tissues probed with anti-G3BP-2 polyclonal antibodies. Some tissues are shown to express both isoforms of G3BP-2 (FIG. 5, panel B) including lung, liver, kidney, stomach and colon (also pancreas and testis, data not shown). Other tissues are restricted to only expressing G3BP-2a (upper band in FIG. 5, panel B) including brain, muscle (small amount of G3BP-2b expression is seen and is presumably caused by the presence of different cell populations within the sample), and heart. Small intestine expresses only MmuG3BP-2b (lower band FIG. 5, panel B) and spleen does not express either protein at detectable levels. Although the general expression of G3BP-1 appears lower than that of G3BP-2 some tissues express abundant levels of G3BP-1 and include lung, kidney and colon. Heart, liver and spleen also express low levels of G3BP-1.

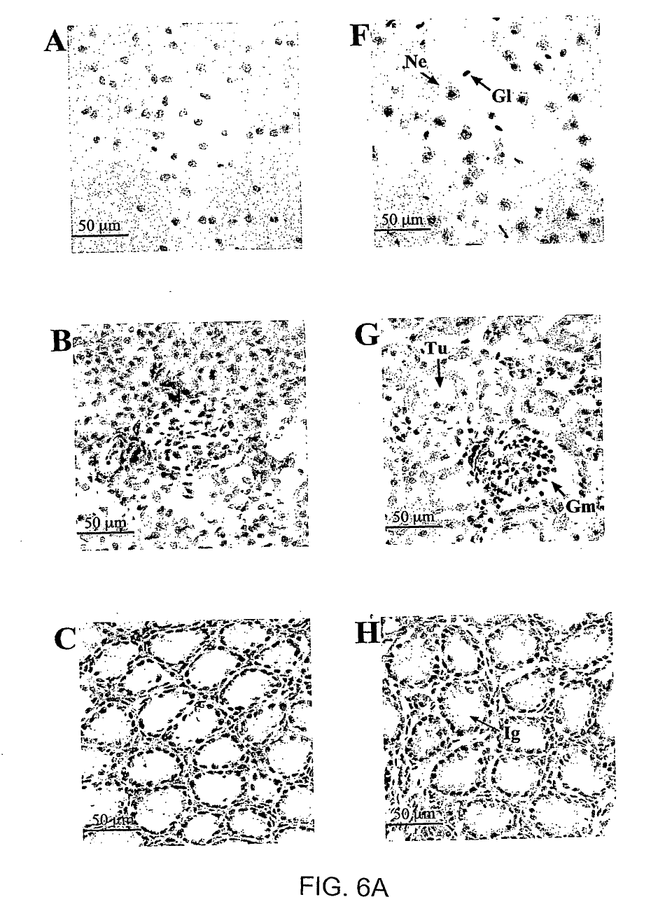

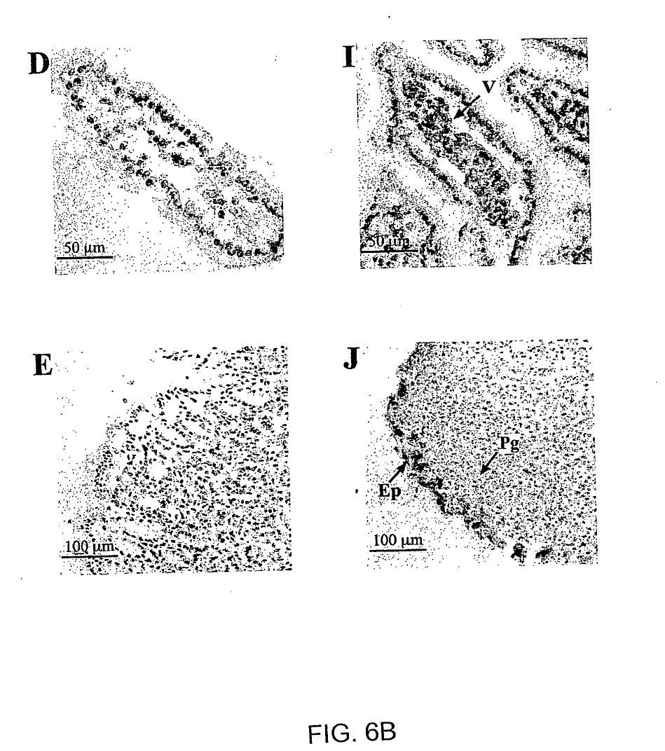

FIG. 6 shows immunohistochemistry results of adult mouse tissues probed with anti-G3BP-1 and anti-G3BP-2 antibodies. Panels A to E are probed with an anti-G3BP-1 antibody whereas panels F to J are probed with an anti-G3BP-2 antibody. All tissue staining was visualised with horse radish peroxidase and sections were counterstained with haematoxylin. Panels A and F are brain (Ne denotes a neurone, GI denotes a glial cell), B and G are kidney (Gm denotes a glomerulus, Tu denotes a tubule), C and H are colon (Ig denotes an intestinal gland) D and I are small intestine (V denotes a villi) and E and J are stomach (Ep denotes epithelial mucus secreting cells and Pg denotes a pyloric gland). All photographs are taken at 100× magnification (bars represent 100 μm) with the exception of stomach (panels E and J), which is displayed at 50× magnification (bars represent 100 μm).

Chromosomal Location of G3BPs

Sequence data obtained from the library clones was used to design G3BP-1 and G3BP-2 specific primers, which were subsequently used on the GeneBridge hybridisation panel to determine the chromosomal localisation of these genes. G3BP-1 mapped to chromosome 5 at 1.51 cR from FB25D10 (lod>3.0) which places the gene between 5q33.1-5q33.3. G3BP-2 mapped to chromosome 4 at 3.36 cR from WI-5565 (lod>3.0) which places the gene between 4q12-4q24. A plasmid artificial chromosome (PAC) library (kindly donated by Dr. P. Ioannou, The Murdoch Institute, Australia) was subsequently screened and isolated clones used to perform fluorescent in-situ hybridisation. The results of the FISH confirmed the genetic location of these genes. In addition to screening the GeneBridge hybridisation panel and the FISH analysis, the human genome sequences in the NCBI databases (http://ncbi.nlm.nih.gov/genome/seq/) were BLAST searched with the cDNA sequences of human G3BP-1 and G3BP-2. The results indicated that there are several chromosomes with matches to either G3BP-1 or G3BP-2, which may represent gene duplications or pseudo-genes. The BLAST analysis on its own was not sufficient to map the G3BPs, however, in conjunction with the FISH and the GeneGridge hybridisation panel results the chromosomal localisation of these genes as indicated above has been confirmed.

A search of the genomic databases (available at http://gdbwww.gdb.org/gdbreports/GeneByChromosome.4.alpha.html and http://gdbwww.gdb.org/gdbreports/GeneByChromosome.5.alpha.html) did not reveal any candidate loci for diseases that may represent genetic defects/polymorphisms in this family of proteins although, there does appear to be some clustering of RNA-binding protein genes on chromosome 5q including, hnRNP A/B (GDB:128837), hnRNP H1 (GDB:5428597), ribosomal protein L7 pseudo gene (GDB:277889), ribosomal protein S14 (GDB:119572), ribosomal protein S17a-like 1 (GDB:119573), ribosomal protein S20A (GDB:119575) and ribosomal protein S20B (GDB:119576). Chromosome 4q also contains several RNA-binding proteins including EIF4EL1 (GDB:126371), G-rich RNA sequence binding factor 1 (GDB:696354), hnRNP D (GDB:9391694), RNA polymerase II polypeptide B (GDB:135034) and ribosomal protein L34 (GDB:9863242). Segment deletions and breakpoint analysis of regions overlapping the chromosomal location of G3BP-1 suggest that this region may be involved in myeloid leukemias (Horrigan et al., 1999) whereas similar studies for chromosome 4q suggest the region containing the G3BP-2 gene may be involved in colorectal adenoma (Wong et al., 1999) and Hodgkin's disease (Atkin 1998). However, there is insufficient data to suggest that any G3BPs are involved in human pathologies that are linked to these regions of the human chromosomes.

Antibodies to G3BP-2

The invention also relates to antibodies against the isolated G3BP-2 polypeptide, fragments, variants and derivatives thereof. A peptide fragment of G3BP-2 may comprise amino acid sequence SATPPPAEPASLPQEPPKPRV [SEQ ID NO: 3], as herein described. Antibodies of the invention may be polyclonal or monoclonal. Well-known protocols applicable to antibody production, purification and use may be found, for example, in Chapter 2 of Coligan et al., CURRENT PROTOCOLS IN IMMUNOLOGY (John Wiley & Sons NY, 1991-1994) and Harlow, E. & Lane, D. Antibodies: A Laboratory Manual, Cold Spring Harbor, Cold Spring Harbor Laboratory, 1988, which are both herein incorporated by reference.

Generally, antibodies of the invention bind to or conjugate with a polypeptide, fragment, variant or derivative of the invention. For example, the antibodies may comprise polyclonal antibodies. Such antibodies may be prepared for example by injecting a polypeptide, fragment, variant or derivative of the invention into a production species, which may include mice or rabbits, to obtain polyclonal antisera. Methods of producing polyclonal antibodies are well known to those skilled in the art. Exemplary protocols which may be used are described for example in Coligan et al., CURRENT PROTOCOLS IN IMMUNOLOGY, supra, and in Harlow & Lane, 1988, supra.

In lieu of the polyclonal antisera obtained in the production species, monoclonal antibodies may be produced using the standard method as for example, described in an article by Köhler & Milstein, 1975, Nature 256, 495, which is herein incorporated by reference, or by more recent modifications thereof as for example, described in Coligan et al., CURRENT PROTOCOLS IN IMMUNOLOGY, supra by immortalizing spleen or other antibody producing cells derived from a production species which has been inoculated with one or more of the polypeptides, fragments, variants or derivatives of the invention.

The invention also includes within its scope antibodies, which comprise Fc or Fab fragments of the polyclonal or monoclonal antibodies referred to above. Alternatively, the antibodies may comprise single chain Fv antibodies (scFvs) against the peptides of the invention. Such scFvs may be prepared, for example, in accordance with the methods described respectively in U.S. Pat. No. 5,091,513, European Patent No 239,400 or the article by Winter & Milstein, 1991, Nature 349 293, which are incorporated herein by reference.

The antibodies of the invention may be used for affinity chromatography in isolating natural or recombinant polypeptides of the invention. For example, reference may be made to immunoaffinity chromatographic procedures described in Chapter 9.5 of Coligan et al., CURRENT PROTOCOLS IN IMMUNOLOGY, supra.

Antibodies may be purified from a suitable biological fluid of the animal by ammonium sulfate fractionation, affinity purification or by other methods well known in the art. Exemplary protocols for antibody purification are given in Sections 10.11 and 11.13 of Ausubel et al., supra, which are herein incorporated by reference.

Immunoreactivity of the antibody against the native or parent polypeptide may be determined by any suitable procedure such as, for example, Western blot.

Mimetics, Agonists and Antagonists

G3BP-2 offers a unique possibility for therapeutics because of its interaction with oncogenic pathways and its unique features, which appear to regulate cell cycle. Of particular interest is an N-terminal NTF2-like domain, which has a surprising host of activities:
(1) The NTF2-like domain appears to regulate nuclear localisation through an interaction with the ran nuclear pore protein.
(2) The expression of a known oncogene, NFκB, through interactions with a Ubiquitin hydrolase, ODE1.
(3) Signal transduction through interactions with GAP¹²⁰

The inventors have also determined that G3BP-2 receives its messages from GAP¹²⁰ through the NTF2-like domain. The NTF2-like domain of G3BP-2 is highly conserved to the entire NTF2 protein. NTF2 is a nuclear pore protein that shuttles into the nucleus through energy dependent interactions with ran. The inventors speculate that G3BP-2 shuttles in and out of the nucleus using the same mechanisms as NTF2 and to this extent they have shown that G3BP-2 interacts with ran. The inventors have also determined that the NTF2-like domain of G3BP-2 interacts with ODE1, a ubiquitin hydrolase and that this interaction can increase the gene expression of NFκB, another protein implicated in tumour progression.

Targeting the NTF2-like domain for anti-cancer therapeutics may inhibit tumour cell proliferation. This would be achieved by blocking ability of G3BP-2 to receive signals from GAP¹²⁰, to block its ability to shuttle mRNA transcripts from the nucleus to the cytoplasm and to block its ability to cause the up-regulation of NFκB.

The invention contemplates agents which may prevent or disrupt formation of polypeptide complexes comprising G3BP-2 and a native or endogenous target polypeptide. Such an agent may be a mimetic, which antagonizes or mimics one or more biological activities of G3BP-2 polypeptides, or homologs thereof.

It will be appreciated that G3BP-2 comprises several recognisable sub-domains (an acid-rich domain, an RNA-recognition motif, an arginine-glycine rich motif and a proline-rich motif). A key to its biological activity as a polypeptide that can facilitate cancer progression may lie in the G3BP-2 N-terminal NTF2-like domain.

The NTF2-like domain is considered to be preferred target for the screening or design of potential G3BP-2 mimetics.

The term “mimetic” is used herein to refer to molecules that are designed to resemble particular functional regions of proteins or peptides, and includes within its scope the terms “agonist”, “analogue” and “antagonist” as are well understood in the art.

An antagonist may be a competitive antagonist or non-competitive antagonist.

It is contemplated that mimetics could be engineered which disrupt or prevent formation of polypeptide complexes between G3BP-2 and endogenous target polypeptides. A mimetic preferably disrupts or prevents formation of a complex between the NTF2-like domain of G3BP-2 and an endogenous target peptide, for example rasGAP¹²⁰. In particular, a polypeptide fragment of rasGAP¹²⁰ comprising the SH3 domain having amino acid sequence [SEQ ID NO: 6] (NCBI accession number: P20936):
VRAILPY TKVPDTDEIS FLKGDMFIVH NELEDGWMWV TNLRTDEQGL IVEDLVEEVG REED

Conversely, it is contemplated that an analogue of the NTF2-like domain of G3BP-2 could be engineered which enables formation of a complex between the analogue and an endogenous native target polypeptide of G3BP-2, thereby competing with G3BP-2 for binding of the endogenous native target. Suitably, the analogue would also bind an endogenous target polypeptide of G3BP-2.

The aforementioned mimetics may be peptides, polypeptides or other organic molecules, preferably small organic molecules, with a desired biological activity and half-life.

Mimetics may be identified by way of screening libraries of molecules such as synthetic chemical libraries, including combinatorial libraries, by methods such as described in Nestler & Liu, 1998, Comb. Chem. High Throughput Screen. 1 113 and Kirkpatrick et al., 1999, Comb. Chem. High Throughput Screen 2 211.

It is also contemplated that libraries of naturally-occurring molecules may be screened by methodology such as reviewed in Kolb, 1998, Prog. Drug. Res. 51 185.

More rational approaches to designing mimetics may employ computer assisted screening of structural databases, computer-assisted modelling, or more traditional biophysical techniques which detect molecular binding interactions, as are well known in the art.

Computer-assisted structural database searching is becoming increasingly utilized as a procedure for identifying mimetics. Database searching methods which, in principle, may be suitable for identifying mimetics, may be found in International Purification WO 97/418232 (directed to producing HIV antigen mimetics), U.S. Pat. No. 5,752,019 and International Publication WO 97/41526 (directed to identifying EPO mimetics), each of which is incorporated herein by reference.

Other methods include a variety of biophysical techniques, which identify molecular interactions. These allow for the screening of candidate molecules according to whether said candidate molecule affects formation of G3BP-2:endogenous target polypeptide complexes, for example. Methods applicable to potentially useful techniques such as competitive radioligand binding assays (see Upton et al., 1999, supra for relevant methods), analytical ultracentrifugation, microcalorimetry, surface plasmon resonance and optical biosensor-based methods are provided in Chapter 20 of CURRENT PROTOCOLS IN PROTEIN SCIENCE Eds. Coligan et al., (John Wiley & Sons, 1997) which is incorporated herein by reference.

Pharmaceutical Compositions

A further feature of the invention is use of the polypeptide, fragment, variant or derivative thereof as actives in a pharmaceutical composition. The actives may be “immunogenic agents” which are capable of eliciting an immune response in an animal. An immunogenic agent may comprise a protein, nucleic acid, vaccine or antigen presenting cell loaded or pulsed with an antigen, or any combination of these agents. The antigen presenting cell may be loaded or pulsed with antigen by contacting the cell with an antigen, for example a protein, polypeptide, fragment, variant or derivative of the invention. The antigen may be internalised within the antigen presenting cell by any suitable means including for example, phagocytosis, micro-injection, engulfing, and the like. The antigen may be combined with any suitable carrier, for example a latex bead, fusion protein, or any other delivery particle commonly used in the art. The antigen presenting cell may be, for example a dendritic cell or any other antigen presenting cell as known in the art of immunology.

A pharmaceutical composition may also comprise an antagonist, which prevents binding between the NTF2-like domain of G3BP-2 and an endogenous binding partner thereof.

Suitably, the pharmaceutical composition comprises a pharmaceutically-acceptable carrier. By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline, and pyrogen-free water.

Any suitable route of administration may be employed for providing a patient with the pharmaceutical composition of the invention. For example, oral, rectal, parenteral, sublingual, buccal, intravenous, intra-articular, intramuscular, intra-dermal, subcutaneous, inhalational, intraocular, intraperitoneal, intracerebroventricular, transdermal and the like may be employed. Intra-muscular and subcutaneous injection is appropriate for administration of immunogenic agents of the present invention.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Pharmaceutical compositions of the present invention suitable for administration may be presented as discrete units such as vials, capsules, sachets or tablets each containing a pre-determined amount of one or more immunogenic agent of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more immunogenic agents as described above with the carrier, which constitutes one or more necessary ingredients. In general, the compositions are prepared by uniformly and intimately admixing the agents of the invention with liquid carriers or finely divided solid carriers or both, and then, if necessary, shaping the product into the desired presentation.

Vaccines

The above compositions may be used as a therapeutic or prophylactic vaccines comprising a polypeptide and/or nucleic acid of the invention, or respective fragments thereof. In one embodiment, the vaccine comprises an immunogenic agent as described above. Preferably, the vaccine prevents or treats breast cancer.

Accordingly, the invention extends to the production of vaccines comprising as actives one or more of the immunogenic agents of the invention. Any suitable procedure is contemplated for producing such vaccines. Exemplary procedures include, for example, those described in NEW GENERATION VACCINES (1997, Levine et al., Marcel Dekker, Inc. New York, Basel Hong Kong) which is incorporated herein by reference.

An immunogenic agent according to the invention can be mixed, conjugated or fused with other antigens, including B or T cell epitopes of other antigens. In addition, it can be conjugated to a carrier as described below.

When a haptenic peptide of the invention is used (i.e., a peptide which reacts with cognate antibodies, but cannot itself elicit an immune response), it can be conjugated with an immunogenic carrier. Useful carriers are well known in the art and include for example: thyroglobulin; albumins such as human serum albumin; toxins, toxoids or any mutant cross reactive material (CRM) of the toxin from tetanus, diptheria, pertussis, Pseudomonas, E. coli, Staphylococcus, and Streptococcus; polyamino acids such as poly(lysine:glutamic acid); influenza; Rotavirus VP6, Parvovirus VP1 and VP2; hepatitis B virus core protein; hepatitis B virus recombinant vaccine and the like. Alternatively, a fragment or epitope of a carrier protein or other immunogenic polypeptide may be used. For example, a haptenic peptide of the invention can be coupled to a T cell epitope of a bacterial toxin, toxoid or CRM. In this regard, reference may be made to U.S. Pat. No. 5,785,973 which is incorporated herein by reference.

The vaccines can also contain a physiologically-acceptable carrier, diluent or excipient such as water, phosphate buffered saline and saline.

The vaccines and immunogenic agents may include an adjuvant as is well known in the art. Suitable adjuvants include, but are not limited to adjuvants for use in human for example SBAS2, SBAS4, QS21 or ISCOMs.

The immunogenic agents of the invention may be expressed by attenuated viral hosts. By “attenuated viral hosts” is meant viral vectors that are either naturally, or have been rendered, substantially avirulent. A virus may be rendered substantially avirulent by any suitable physical (e.g., heat treatment) or chemical means (e.g., formaldehyde treatment). By “substantially avirulent” is meant a virus whose infectivity has been destroyed. Ideally, the infectivity of the virus is destroyed without affecting the polypeptides that carry the immunogenicity of the virus. From the foregoing, it will be appreciated that attenuated viral hosts may comprise live viruses or inactivated viruses.

Attenuated viral hosts which may be useful in a vaccine according to the invention may comprise viral vectors inclusive of adenovirus, cytomegalovirus and preferably pox viruses such as vaccinia (see for example Paoletti and Panicali, U.S. Pat. No. 4,603,112 which is incorporated herein by reference) and attenuated Salmonella strains (see for example Stocker, U.S. Pat. No. 4,550,081 which is herein incorporated by reference). Live vaccines are particularly advantageous because they lead to a prolonged stimulus that can confer substantially long-lasting immunity.

Multivalent vaccines can be prepared from one or more different epitopes of G3BP-2.

A recombinant vaccinia virus may be prepared to express a nucleic acid according to the invention. Upon introduction into a host, the recombinant vaccinia virus expresses the immunogenic agent, and thereby elicits a host CTL response. For example, reference may be made to U.S. Pat. No. 4,722,848, incorporated herein by reference, which describes vaccinia vectors and methods useful in immunization protocols.

A wide variety of other vectors useful for therapeutic administration or immunization with the immunogenic agents of the invention will be apparent to those skilled in the art from the present disclosure.

The nucleic acid of the invention may be used as a vaccine in the form of a “naked DNA” vaccine as is known in the art. For example, an expression vector of the invention may be introduced into a mammal, where it causes production of a polypeptide in vivo, against which the host mounts an immune response as for example described in Barry, M. et al., (1995, Nature, 377:632-635) which is hereby incorporated herein by reference.

Dendritic Cell Therapy

“Dendritic cells” (DC) are antigen presenting cells capable of initiating an antigen-specific T-cell response in an animal. DC's may be isolated from various locations of an animal's body, including peripheral blood. Methods for in vitro proliferation and expansion of DC precursors have been described, for example in U.S. Pat. No. 5,994,126, incorporated herein by reference. Also described is a method for producing mature dendritic cells in culture from proliferating dendritic cell precursors.

“Dendritic cell therapy” refers to therapeutic cancer vaccines or cellular vaccines used for tumour immunotherapy as a method for treating cancer. Dendritic cell therapy typically involves isolating DC from a patient, culturing the isolated DC in the presence of a tumour-associated antigen (TAA) thereby contacting the DC with a TAA (“antigen loading or pulsing”), and administering the antigen loaded DC's to the patient. Other methods for antigen loading an isolated DC include transfecting, micro-injecting, calcium phosphate transfection, DEAE-transfection, electroporation or otherwise introducing an isolated nucleic acid encoding a tumour-associated antigen into the isolated DC. Common method for introducing nucleic acids into a cell are described in Chapter 9 of CURRENT PROTOCOLS IN MOLECULAR BIOLOGY Eds. Ausubel et al., (John Wiley & Sons, Inc. 1995-1999), incorporated herein by reference. Preferably, the TAA is G3BP-2, fragment, variant, homolog or derivative thereof. The nucleic acid may be DNA or RNA. The nucleic acid may be transiently or stably express the TAA as is known in the art.

Methods for loading or pulsing dendritic cells with an antigen are described in (Meidenbauer et al, 2001, Biol Chem 4 507; Rains, et al, 2001, Hepatogastroenterology 38 347; Nestle, 2000, Oncogene 56 6673; Gilboa and Lyerly, 1998, Cancer Immunotherapy 46 82) incorporated herein by reference.

U.S. Pat. No. 5,788,963, incorporated herein by reference, describes methods and compositions for use of human dendritic cells to activate T-cells for immunotherapeutic responses against primary and metastatic prostate cancer. In one embodiment isolated DC are exposed in vitro to a prostate cancer antigen before administration to a patient.

In one embodiment of the present invention, a pharmaceutical composition comprises DC's antigen load with G3BP-2 polypeptide, fragment, variant or derivative thereof in accordance with the invention. In another embodiment of the present, the DC's are transfected with a nucleic acid encoding a G3BP-2 polypeptide, fragment, variant or derivative thereof. Suitable G3BP-2 protein fragments for use with DC cell therapy are set forth as SEQ ID NOS: 1 and 2. It will be appreciated by a skilled person that antigen presenting cells other than DC may be used and that use of DC is merely preferred.

Preparation of Immunoreactive Fragments

The invention also extends to a method of identifying an immunoreactive fragment of a polypeptide, variant or derivatives according to the invention. This method essentially comprises generating a fragment of the polypeptide, variant or derivative, administering the fragment to a mammal; and detecting an immune response in the mammal. Such response may include production of elements which specifically bind G3BP-2, respective variant, or derivative thereof, including NTF2-like domain, which may provide a protective effect against breast cancer.

Prior to testing a particular fragment for immunoreactivity in the above method, a variety of predictive methods may be used to deduce whether a particular fragment can be used to obtain an antibody that cross-reacts with the native antigen. These predictive methods may be based on amino-terminal or carboxy-terminal sequence as for example described in Chapter 11.14 of Ausubel et al., supra. Alternatively, or in addition, these predictive methods may be based on predictions of hydrophilicity as for example described by Kyte & Doolittle 1982, J. Mol. Biol. 157 105 and Hopp & Woods, 1983, Mol. Immunol. 20 483) which are incorporated by reference herein, or predictions of secondary structure as for example described by Choo & Fasman, 1978, Ann. Rev. Biochem. 47 251), which is incorporated herein by reference.

Generally, a peptide fragment consisting of 10 to 15 residues provides optimal results. Peptides as small as 6 or as large as 20 residues have worked successfully. Such peptide fragments may then be chemically coupled to a carrier molecule such as keyhole limpet hemocyanin (KLH) or bovine serum albumin (BSA) as for example described in Sections 11.14 and 11.15 of Ausubel et al., supra).

The peptides may be used to immunize an animal as for example discussed above. Antibody titers against the native or parent polypeptide from which the peptide was selected may then be determined by, for example, radioimmunoassay or ELISA as for instance described in Sections 11.16 and 11.14 of Ausubel et al., supra.

Immunoreactive protein fragments in the context of the Major Histocompatibility Complex (MHC) may be determined using methods well known in the art including those described by Schultze and Vonderheide, 2001, incorporated herein by reference.

Detection Kits

The present invention also provides a kit for detection of G3BP-2 in a biological sample. A kit will contain one or more particular agents described above depending upon the nature of the test method employed. In this regard, the kits may include one or more of a polypeptide, fragment, variant, derivative, antibody, antibody fragment or nucleic acid according to the invention. The kits may also optionally include appropriate reagents for detection of labels, positive and negative controls, washing solutions, dilution buffers and the like. For example, an antibody-based detection kit may include (i) a polypeptide, or fragment or variant thereof according to the invention (which may be used as a positive control), (ii) an antibody according to the invention (preferably a monoclonal antibody) which binds to G3BP-2 or fragment thereof in (i), and (iii) a suitable means for detecting a complex formed between a target (eg. G3BP-2 in a sample) and the antibody in (ii), the detection means may include, for example colloidal gold. Suitable antibodies for use in a detection kit include those described herein in relation to Western blots and immunohistochemistry.

A detection kit may also be nucleic acid based. Such a detection kit may include the step of amplifying a nucleic acid from the test sample obtained from an animal using techniques such as Polymerase Chain Reaction (PCR) or other known amplification method known in the art. Useful PCR primers may include those set forth herein as SEQ ID NOS: 16-21. The nucleic acid may be RNA or DNA. The test sample is preferably breast tissue isolated from a patient.

Throughout this specification, unless the context requires otherwise, the words “comprise”, “comprises” and “comprising” will be understood to imply the inclusion of a stated integer or group of integers but not the exclusion of any other integer or group of integers.

In order that the invention may be readily understood and put into practical effect, particular preferred embodiments will now be described by way of the following non-limiting examples.

### EXAMPLE 1

Cloning, Sequence Homology and Structural Homology

PCR and Subcloning

PCR reactions to amplify probes or for hybrid mapping were carried out using 1.1 units of Tth Plus DNA Polymerase fragment (Biotech International) and buffer supplied by the manufacturer (Biotech International) and contained 100 ng template DNA and 50 pmol of each appropriate primer. Cycling conditions were: denaturation of DNA at 94° C. for 1 min, annealing at 65° C. for 1 min and extension at 72° C. for 1 min for 25 cycles. Primers that can be used to amplify either full length G3BPs or the NTF2-like domain of the G3BPs:

Full length human G3BP-1 is amplified using primers G3BP-2met with G3BP-1stop; full length human G3BP-2 is amplified using primers G3BP-2met with G3BP-2stop; the NTF2-like domain of human G3BP-1 is amplified using primers G3BP-1 met with G3BP-1 ntf; and the NTF2-like domain of human G3BP-2 is amplified using primers G3BP-2met with G3BP-2ntf.

Primer Sequences:

| | |
|---|---|
| G3BP-1met | |
| atggtgatggagaagcctagtcccctgctggt | [SEQ ID NO:16] |
| G3BP-2met | |
| atggttatggagaagcccagtccg | [SEQ ID NO:17] |
| G3BP-2met | |
| atcaagttcaggctcagaatcacc | [SEQ ID NO:18] |
| G3BP-1met | |
| ctgaggctcagtgacaaacccaac | [SEQ ID NO:19] |
| G3BP-2stop | |
| gcttcagcgacgctgtcctgtgaagc | [SEQ ID NO:20] |
| G3BP-1stop | |
| ctgccgtggcgcaagcccccttcc | [SEQ ID NO:21] |

Library Screening and Phage Isolation

Plasmid preparations, to be used as probes, library screening or sequencing, were made as described by Sambrook et al., 1989 (Sambrook et al., 1989). The libraries, late-primitive-streak stage mouse embryo cDNA (Kennedy et al. 1997) and a foetal human brain (cDNA kindly donated by Dr. T. Cox, University of Adelaide, Australia) were plated out at approximately 25 000 pfu/130 mm plate and duplicate filters (Hybond-N, Amersham) made from 21 plates. Library screening was performed as described by Sambrook et al., 1989 (Sambrook et al. 1989) with radioactive probes prepared using Amersham's Megaprime DNA labeling system according to the manufacturer's instructions. Hybridization conditions were as follows: 50% formamide, 5×SSC, 20 mM Tris.HCl pH 7.6, 1× Denhardt's solution and 0.1% SDS at 42° C. A minimum of 3×20 min washes of hybridized filters were performed in 0.2×SSC, 0.1% SDS at 65° C. cDNAs from purified plaques were subcloned into pBluescript SK (Stratagene).

### EXAMPLE 2

Protein Expression in Insect Cells

Sub Cloning and Expression of Proteins Using Baculovirus

Full length cDNAs encoding for MmuG3BP-2a and 2b were excised from pBluescript using EagI (−85 bp from met start codon) and AccI (+140 bp from the stop codon) and subsequently end filled using Klenow fragment polymerase. These cDNAs were subcloned into SmaI linearised pBacPAK9 (Clontech) and transformed into competent DH5′α E. coli cells. The orientation of the inserts were checked by PCR. Plasmids containing the inserts in the correct orientation were transfected into Spodoptera frugiperda IPLB-Sf21 (Sf21) cells with Bsu36I digested BacPAK6 viral expression vector according to the manufacturers instructions (Clontech #K1601-1). Recombinant virus plaques were selected from an Sf21 monolayer and once again screened by PCR. Virus containing the coding cDNAs were amplified in Sf21 cells and total cell lysates visualised on polyacrylamide gels for expression of proteins.

### EXAMPLE 3

Mapping of G3BP-rasGAP¹²⁰ Interactions

Fusion Protein Constructs and Expression

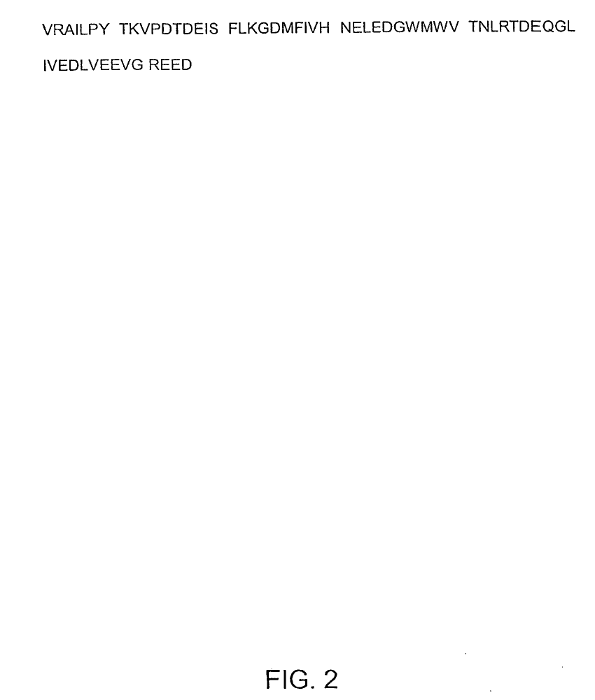

Truncated cDNAs representing either N-terminal or C-Terminal sequences of G3BP-1 and G3BP-2a (FIG. 2) were subcloned into bacterial GST-fusion expression vectors (pGex, Pharmacia) so that the recombinant fusion proteins could be expressed and used in bead binding assays (see below) to identify the sub-domains of G3BP that interact with the SH3 domain of rasGAP¹²⁰. The vectors containing the recombinant fusion protein construct were transformed into competent DH5′ E. Coli and expressed by IPTG induction. To collect the recombinant proteins the cells were washed and resuspended in PBS and sonicated to release the fusion proteins.

The N-terminal domain of rasGAP¹²⁰ (kindly provided by Prof. I.G. Macara, University of Virginia) containing amino acids 1-356 which includes the full SH3 domain of rasGAP¹²⁰ was subcloned into proEX HT bacterial expression vector (Life Technologies) and expressed as described above.

Bead Binding Assays

Glutathione columns (Pharmacia) were blocked overnight at 4° C. in 1× binding buffer A (50 mM HEPES pH 7.4, 100 mM NaCl, 5 mM MgCl₂, 50 mg/ml BSA, 1 mM DTT, 1 mM PMFS and protease inhibitors (Protease inhibitor cocktail, Roche Diagnostics, Australia). The following day the columns were washed twice with binding buffer A. Bacterial lysates containing either expressed GST alone or GST fusion proteins were diluted 1:1 with 2× binding buffer A and added to the equilibrated GST columns, the columns were gently rocked overnight at 4° C. To remove excess proteins the columns were washed twice in 1× binding buffer A. Bacterial lysates containing the His-tagged N-terminal of rasGAP¹²⁰ protein were combined with an equal volume of 2× binding buffer A containing 200 mg/ml BSA and 0.1% Tween. In addition to the high concentration of BSA, which is used to block non-specific protein-protein interactions, Ethidium bromide was added to a final concentration of 25 ng/ml to abrogate non-specific interactions caused by excess bacterial genomic DNA and bacterial RNA. This mix was then added to the pre-bound GST columns (above) and allowed to bind for 2 hrs at 4° C. The columns were then washed three times with binding buffer B (50 mM HEPES pH 7.4, 200 mM NaCl, 5 mM MgCl₂, 1 mM DTT, 1 mM PMFS and protease inhibitors).

Western Analysis of Protein-Protein Interactions

One μl of the GST beads complexed with proteins, as described above, was added to an equal volume of 2×PAGE loading dye, heated for 5 mins at 95° C. and loaded onto a 7.5% Polyacrylamide gel and run at 100V for 3 hours. The proteins were transferred to Nitrocellulose and probed with anti-His antibodies (Tetra-His antibody, Qiagen). Columns, which were bound with GST alone, were used as negative controls to ensure no non-specific His-tagged N-terminal rasGAP¹²⁰ remained associated with the columns. To ensure that the GST columns had been saturated with the appropriate GST-G3BP peptides all experiments were probed with anti-GST to confirm that the columns contained the “bait” peptide (data not shown).

Protein Purification

500 ml of Sf21 cells at a concentration of 1.2 to 2.0×10⁶ cells/ml cells were infected with 60 ml of baculovirus (6.6×10⁸ to 1.6×10⁹ pfu/ml) containing cDNAs for expression of proteins and incubated at 28° C. for 4 days in an orbital incubator. The cells were harvested and washed twice at 4° C. with PBS, lysed in 50 ml of HNTG lysis buffer by 30 sec vortexing and gentle rocking for 30 min at 4° C. and cleared by centrifugation at 9500 rpm×10 min at 4° C. The final salt concentration was adjusted to 30 mM NaCl by addition of NaCl free and triton X-100 free HNTG lysis buffer (containing protease inhibitors) and incubated overnight on a rotating mixer at 4° C. with pre-equilibrated heparin sepharose CL-6B (Pharmacia biotech #17-0467-01) at a concentration of 15 mg of protein/ml of heparin sepharose. The gel was washed in 50 mM Hepes pH 7.5, 10% glycerol and packed into a column (Pharmacia XK-26). The column was subjected to a 30 mM to 1.0 M NaCl gradient over 120 min at a flow rate of 0.83 ml/min using a Pharmacia FPLC system. 1.5 ml samples were collected and assayed for MmuG3BP by separation on polyacrylamide gels and visualised by coomassie blue staining or Western blot analysis using the 663 antibody.

Fractions containing the recombinant G3BP proteins were pooled and again diluted to a final 60 mM NaCl concentration. The pooled samples were incubated with agarose-polyribouridylic acid AGPoly(U), type 6 (Pharmacia biotech #27-5535) at a concentration of 1.5 mg of protein/ml of gel overnight at 4° C. on a rotating mixer. The following morning the gel was washed with 50 mM Hepes pH 7.5, 10% glycerol, packed into a glass column (Pharmacia XK-16) and subjected to a 60 mM to 1 M NaCl gradient at a flow rate of 0.33 ml/min as described above. 0.5 ml fractions were collected, assayed and pooled as described above. Pooled samples were diluted to 30 mM NaCl as described above and loaded onto a mono S HR 5/5 ion exchanger column (Pharmacia #17-0547-01) at a flow rate of 0.3 ml/min and subjected to a 30 mM to 1 M NaCl gradient. 0.5 ml fractions were collected and assayed as described above.

### EXAMPLE 4

G3BP-2 Anti-Sera

Polyclonal Antibody Production

Affinity purified antibodies to G3BP-2 were obtained from an antiserum raised against an internal peptide sequence (SATPPPAEPASLPQEPPKPRV). Polyclonal antibodies raised against G3BP-1 have been described elsewhere (Parker et al. 1996) and a monoclonal G3BP-1 antibody is commercially available as (BD Biosciences, Sydney, AUS).

G3BP-2 Antibody Specificity

The specificity of the G3BP-2 antibody was assessed by testing its ability to bind human recombinant GST fusion G3BP-1, G3BP-2a, G3BP-2b and G3BP-2a truncations. LB/Amp agar plates were inoculated with E. coli transformed with pGEX vectors (Amersham Biosciences, GST gene fusion system) containing four alternative truncations of G3BP-2a (N1, N2, C1 and C2), as well as full length G3BP-1, 2a and 2b, as previously described in Kennedy et al. (2001). One colony from each plate was used to inoculate 5 ml of LB/Amp broth, which was incubated at 37° C. overnight. Isolation of the GST fusion proteins was performed by glutathione sepharose affinity chromatography from IPTG-induced cultures as per the manufacturer's instructions (Amersham Biosciences). Following elution with glutathione, the solutions were spun at 5000 rpm for 5 minutes and the supernatant was dialysed in PBS.

Purified recombinant G3BP-1, G3BP-2a, G3BP-2b, and the four truncations of G3BP-2a were resolved by 12% SDS-PAGE and transferred to PVDF (Millipore) and incubated with anti-G3BP-2 antibody. Proteins were visualised by HRP conjugated anti-rabbit antibodies using an ECL system (Amersham Biosciences).

Cell Extracts, SDS-PAGE and Western Blotting

The expression of G3BP in human cell lines was examined by Western blot. Cells were maintained in vitro in DMEM supplemented with 10% FCS and harvested by trypsinisation, washed twice with PBS and resuspended in HNTG buffer (50 mM Hepes, pH 7.5, 150 mM NaCl, 1% Triton X-100, 10% glycerol, 1 mM MgCl₂, 1 mM EGTA, 1 mM Na₃VO₄, 10 mM Na₄P₂O₇, 10 mM NaF, 1 mM PMSF and 1× mammalian protease inhibitor cocktail # P8340 (Sigma, Castle Hill, AUS). Lysates were cleared by centrifugation at 15 000 rpm for 10 minutes and the protein concentration was determined using the Pierce BCA Protein Assay (Rockford, USA). A total of 75 μg of protein was resolved by 8% SDS-PAGE and transferred to an Immobilon-P PVDF membrane (Millipore, Sydney, AUS) for Western analysis using the antibodies described above. Proteins were visualised by horseradish peroxidase (HRP) conjugated anti-rabbit or mouse antibodies using an ECL system (Amersham Biosciences, Sydney, AUS).

Antibodies Used for Immunohistochemistry were Highly Specific

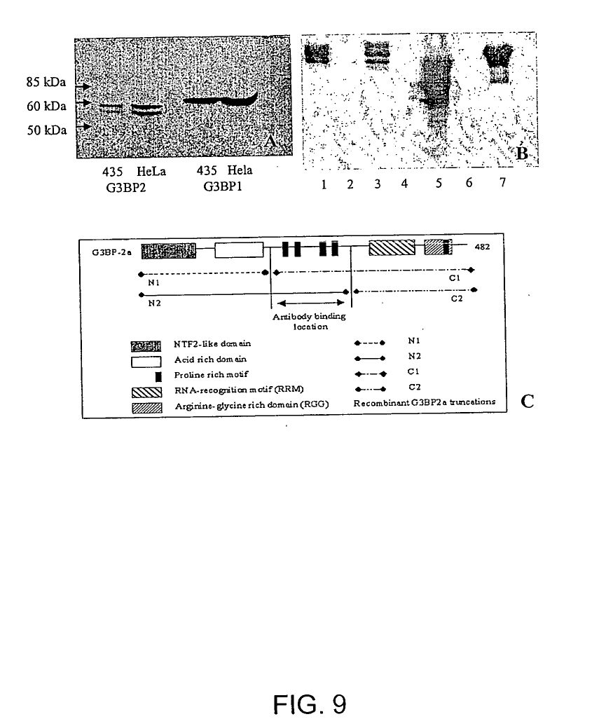

To assess antibody specificity and the expression of G3BPs in a range of cell types, the breast cancer cell line, MDA-MB-435 and the cervical cancer cell line, HeLa were lysed and equal amounts of protein were resolved by SDS-PAGE. The samples were transferred to a membrane and analysed by Western blotting. The commercial monoclonal G3BP-1 antibody (BD Biosciences) was used to assess G3BP-1 expression, while the polyclonal G3BP-2 antibody (Kennedy et al. 2001) was used to examine the expression of G3BP-2 in these cell lines. As illustrated in FIG. 9, Panel A, it is apparent that both cell lines express significant levels of G3BP-1, present as a single distinct band, and G3BP-2, present as two distinct bands, representing the two different isoforms. The same expression patterns were seen in the immortalised human cell line HEK 293T (data not shown). There did not appear to be any significant cross-reactivity. However, there was some variation between the relative masses of G3BP-1, 2a and 2b according to amino acid sequence, and the apparent masses of the proteins as determined by PAGE. According to the data, G3BP-2a should resolve at a point just above G3BP-1, but it actually appears just below G3BP-1.

The specificity of the polyclonal G3BP-2 antibody was further examined. It was tested against recombinant GST-fusion G3BP-1, 2a and 2b as well as several different truncated forms of G3BP-2. As presented in FIG. 9, Panel B, the anti-G3BP-2 antibody specifically binds to recombinant G3BP-2a and G3BP-2b (lanes 3 and 1, respectively) while it does not bind to G3BP-1 (lane 2). The antibody bound two of the recombinant G3BP-2a truncations (lanes 5 and 7) but it did not bind the short C-terminal or N-terminal truncations (Lanes 4 and 6). This indicated that the region that the antibody binds to is the central domain as shown in FIG. 9, Panel C. It was found that the G3BP-2 antibody is specific for G3BP-2a and 2b and binds the central region of the protein only. Despite excessively high protein loads (3 μg of recombinant protein per lane), the antibody did not cross-react with G3BP-1 or the shorter of the G3BP-2 N-terminal (N1) or C-terminal (C2) truncations.

### EXAMPLE 5

G3BPs have a Tissue Specific Expression

Western Blotting

Proteins were fractionated by sodium dodecyl sulfate (SDS)-polyacrylamide gel electrophoresis (7.5% gel) using the method of Laemmli and transferred onto polyvinylidene difloride (PVDF) membrane (Millipore Corp.) in a Bio-Rad Trans-blot Cell using a transfer buffer containing 25 mM Tris pH 8.3, 192 mM Glycine and 15% methanol. Electroblotting was carried out at 100 volts overnight at 4° C. The blot was blocked by incubation in 20 mM Tris-HCl pH 7.5, 150 mM NaCl, 0.1% Tween-20 containing 10% skim milk (blocking solution) at room temperature for 1 hour, followed by incubation with the primary antibody, anti-G3BP-1 (diluted 1:500) or anti-G3BP-2 (diluted 1:4000) in blocking solution, at 4° C. overnight. The blot was washed 3 times in blocking solution for 10 minutes and subsequently incubated for 2 hours at 37° C. in the secondary antibody which was an anti-rabbit conjugated with horseradish peroxidase (BioRad), diluted 1:10000 in blocking solution.

Total Protein Cell and Tissue Lysates

Cells and tissues were washed twice with ice cold phosphate-buffered saline (PBS) and solubilised or homogenised with a dounce homogeniser in HNTG lysis buffer consisting of 50 mM HEPES, pH 7.5, 150 mM NaCl, 1% Triton X-100, 10% glycerol, 1 mM MgCl₂, 1 mM EGTA, phosphatase inhibitors (1 mM Na₃VO₄, 10 mM Na₄P₂O₇ and 10 mM NaF) and protease inhibitors (1 μg of leupeptin per ml, 1 μg of trypsin inhibitor per ml, 1 μg of pepstatin A, 2 μg of aprotinin per ml, 10 μg benzamidine per ml, 1 mM phenylmethylsulfonyl floride, 1 μg of antipain per ml, 1 μg of chymostatin per ml). Lysates were cleared by centrifugation at 15 000 rpm for 10 min and the protein concentration determined by the Bradford dye-binding procedure using Bio-Rads Protein Assay (# 500-0001).

Immunohistochemistry

Immunohistochemistry was used to analyse the degree of cell specificity of the G3BP-1 and G3BP-2 expression. Until isoform specific antibodies are raised against G3BP-2a and G3BP-2b it is not possible to distinguish these isoforms in immunohistochemistry, however, in some tissues a it can be determined which specific isoform is being expressed by comparison to the Western blot data (as herein described).

FIGS. 6A and 6B show a cross section of results from some of the tissues studied. Panels A to E are probed with anti-G3BP-1 antibodies whereas panels F to J are probed with anti-G3BP-2 antibodies. Panels A and F show a comparison of adult mouse brain. As determined by Western analysis (FIG. 5, panel A), brain does not express G3BP-1 (FIG. 6, panel A) however, a sub-population of cells express G3BP-2a (panel F).

The inventors determined by cell morphology and double staining with a neural marker (data not shown) that the G3BP-2a positive cells are neural cells (panel F, labeled Ne) and that the negative cells are glial cells (panel F, labeled GI). In the kidney (panels B and C) G3BP-1 appears to be expressed in interstitial cells or a sub-population of tubules (panel B) whereas G3BP-2 is expressed at low levels in all tubules (labeled Tu in panel G). Neither G3BP-1 nor G3BP-2 are expressed in the glomerulus (labeled Gm in panel G). The colon (panels C and H) shows that G3BP-1 is expressed at the periphery of the intestinal glands or possibly in interstitial cells whereas G3BP-2 is expressed in the lumen of the intestinal glands (labeled Ig in panels H and I). G3BP-2 is also expressed at high levels in the villi of the small intestine (FIG. 6 panel I) whereas G3BP-1 (FIG. 6 panel D) was not detected at levels above the background staining of the negative control. Once again no detectable staining was observed for G3BP-1 in stomach (FIG. 6 panel E) whereas G3BP-2 (presumably G3BP-2b only from the Western blot data) appears to be expressed in the mucus secreting cells of the stomach lumen (labeled Ep in panel J) and the internal surface of the pyloric glands (labeled Pg in panel J). Other tissues examined by immunohistochemistry include heart, liver and spleen (data not shown). Heart and liver showed a general low level expression of G3BP-1 and G3BP-2 whereas spleen was negative for G3BP-2 and showed a cell specific staining of G3BP-1. It is still to be determined what types of cells constitute the G3BP-1 expressing islands observed within the spleen.

Frozen mouse tissue sections (10 μm thickness) were fixed to Histogrip treated slides (SuperFrost Plus microscope slides, Menzel-Glaser, Germany) and air-dried overnight at room temperature. Sections were fixed for 5 minutes in 50% Chloroform, 50% Acetone, air dried and rehydrated in Tris-buffered saline (TBS) (25 mM Tris, 137 mM NaCl, pH 7.4). Nonspecific antibody binding was inhibited by incubation with TBS containing 4% skim milk powder for 15 minutes followed by an additional 20 min incubation in TBS containing 10% normal goat serum (Gibco). Sections were then incubated overnight with either anti-G3BP-1 (diluted 1:300) or an anti-G3BP-2 (diluted 1:2000). Excess antibody was removed by washing in TBS (3×5 min) and prediluted horseradish peroxidase (HRP) labeled anti-rabbit immunoglobulins (Envision) was applied for 30 minutes. Sections were then washed with TBS (3×5 min) and colour was developed in 3,3′-diaminobenzidine with H₂O₂ (Zymed) as a substrate for 2 minutes. Sections were washed with gently running tap water for 10 minutes to remove excess chromogen, lightly counterstained in Mayers' haematoxylin, dehydrated through ascending graded alcohols, cleared in xylene, then mounted using DPX (Harlow and Lane 1988).

### EXAMPLE 6

Chromosomal Location of G3BPs

Fluorescence In Situ Hybridisation of PACs

Fluorescence in situ hybridisation (FISH) was performed on peripheral human metaphase chromosomes. PAC DNA was biotin-14dATP-labelled by nick translation using the BioNick labeling system (Life Technologies). Chromosome preparation and FISH conditions were as described previously (Wicking et al., 1995). Slides were analysed using an Olympus BH2 fluorescent microscope.

Chromosomal Mapping

Two HsaG3BP-1 specific primers, 5′ GGAGGCATGGTGCAGAAACCA [SEQ ID NO: 12] and 5′ CAGGAAAGGGAAGAGAGGGAG [SEQ ID NO: 13] and two HsaG3BP-2 specific primers, 5′ GTCTTGGCAGTGGTACATTAT [SEQ ID NO: 14] and 5′ AGTTCACTTTGTCGTAGATAGTTTAAG [SEQ ID NO: 15] were used to amplify specific templates from human genomic DNA and were subsequently used on the Genebridge4 Hybrid panel to identify positives. This data was then processed by the online mapping software available through the Whitehead Institute/MIT Center for Genome research (http://carbon.wi.mit.edu).

### EXAMPLE 7

Antagonists of G3BP-2

An antagonist which prevents or disrupts G3BP-2 binding with its endogenous target may be useful in preventing or treating breast cancer. An antagonist may mimic either the NTF2-like domain of G3BP-2 or mimic an endogenous target which binds to the NTF2-like domain. For example, the SH3 domain of rasGAP¹²⁰ could be used as a peptide antagonist for blocking the activity of G3BP-2 in breast cancers. The amino acid sequence of the SH3 domain of rasGAP¹²⁰ is:

| | |
|---|---|
| VRAILPY TKVPDTDEIS FLKGDMFIVH | [SEQ ID NO:6] |
| NELEDGWMWV TNLRTDEQGL IVEDLVEEVG REED | |

The NCBI accession number: P20936 for the above sequence. The antagonists may be a polypeptide, but may also be a non-peptide molecule which is capable of acting as an antagonist.

Mimetics may be identified by way of screening libraries of molecules such as synthetic chemical libraries, including combinatorial libraries, by methods such as described in Nestler & Liu, 1998, supra and Kirkpatrick et al., 1999, supra. Libraries of naturally-occurring molecules may be screened by methodology such as reviewed in Kolb, 1998, supra.

Three-dimensional (3D) structural modelling by homology can be used to assign a 3D structure to the NTF2-like domain of G3BP-2 based on structural information from the known crystal structure of the NTF-2 polypeptide. Methods for 3D structural modelling by homology is described in Blundell et al, 1987, Nature 326 347, herein incorporated by reference. An antagonist that interacts with the NTF2-like domain may be designed based on structural modelling by homology.

Mimetics may be designed using computer assisted screening of structural databases, computer-assisted modelling, or more traditional biophysical techniques which detect molecular binding interactions, as are well known in the art.

Other methods include a variety of biophysical techniques which identify molecular interactions. These methods may screen candidate molecules according to whether the candidate molecule affects formation of G3BP-2:endogenous target polypeptide complexes. Methods applicable to potentially useful techniques such as competitive radioligand binding assays (see Upton et al., 1999, supra for relevant methods), analytical ultracentrifugation, microcalorimetry, surface plasmon resonance and optical biosensor-based methods are provided in Chapter 20 of CURRENT PROTOCOLS IN PROTEIN SCIENCE Eds. Coligan et al., (John Wiley & Sons, 1997) which is incorporated herein by reference.

### EXAMPLE 8

Diagnosis of Breast Cancer

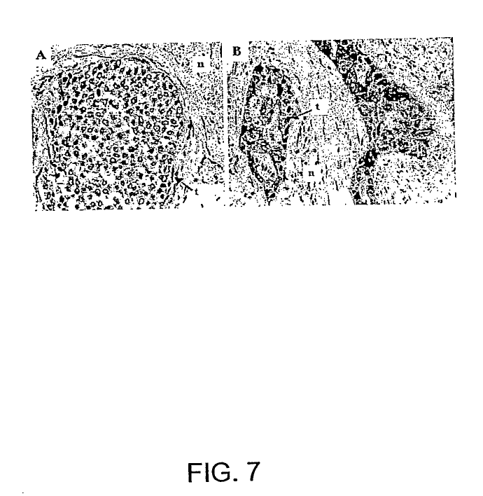

G3BP-2 may be useful for diagnosing breast cancer in an individual. In one embodiment, the method may include the steps of: (i) assaying a test sample obtained from the mammal for expression of G3BP-2 polypeptide; (ii) comparing G3BP-2 expression from the test sample with expression in a normal sample from a normal mammal; and (iii) diagnosing the mammal with a likelihood of breast cancer if the expression of G3BP-2 in the test sample is different than the normal sample. The term different refers to at least a detectable difference either by aided or unaided means. For example, an unaided means includes a person visually comparing a difference in relative apparent abundance of protein, such as a thicker or darker “band” on a Western blot or darker well of an ELISA. Aided means includes for example, use of a microscope to assess antibody binding of a tissue section or an apparatus that is capable is detecting and measuring a difference in protein amount, for example an ELISA plate reader or FACS. The method for diagnosing breast cancer may include the step of detecting a G3BP-2 polypeptide, or fragment thereof, in the test sample using an antibody which binds to the G3BP-2 polypeptide, or fragment thereof. The antibody described herein, for example as used in FIGS. 7-9, may be useful in a diagnostic kit. The antibody may be a polyclonal or monoclonal antibody.

The method for diagnosing breast cancer may include methods of detecting a polypeptide, for example Western blot analysis, ELISA, FACS analysis and immunohistochemistry as is commonly known in the art. Examples of Western blot analysis and immunohistochemistry as described herein may be useful in detecting G3BP-2 polypeptide, fragment, homolog or derivative thereof. Western blot analysis is useful in determining expression of different isoforms of G3BP-2, ie. G3BP-2a and G3BP-2b, which are distinguishable by size, as shown for example in FIG. 5. Immunohistochemistry is useful in determining cellular and subcellular localisation of G3BP-2, as shown for example in FIGS. 6A and 6B. An antibody which specifically binds to either G3BP-2a or G3BP-2b is useful in determining expression respective G3BP-2 isoform.

Detection of G3BP-2 Protein in Human Cancer by Immunohistochemistry

Patients

Fifty-nine cases of invasive breast carcinoma diagnosed at the Department of Pathology, Royal Brisbane Hospital, between 1981 and 1990 were randomly selected. Archival paraffin blocks were accessed subject to ethics approval from the Royal Brisbane Hospital and had been previously characterised as part of a larger study of MUC1 epithelial mucin expression (McGuckin et al. 1995). Histological classification and grading of the tumours was performed in accordance with the Nottingham modification of the Bloom and Richardson system (Elston & Ellis 1990). Data including nodal status and oestrogen receptor (ER) status as determined by biochemical dextran-coated charcoal method were obtained from clinical charts and pathology records.

Immunohistochemistry of Breast Cancer Sections

Breast tumour sections (3-4 μm) were affixed to adhesive slides and dried at 37° C. overnight. The sections were dewaxed and rehydrated through descending graded alcohols to deionised water using standard protocols. Sections to be stained for G3BP-1 were subjected to antigen heat retrieval by autoclaving at 120° C. for 20 minutes in 1 mM EDTA, pH 8.0. G3BP-2 samples were subjected to antigen heat retrieval by boiling in 0.1M Tris-HCl, pH 9.0-9.2, for 5 minutes in a microwave, and repeating the process using fresh Tris-HCl buffer. All sections were allowed to cool to room temperature (20-30 min) and then washed in Tris buffered saline, pH 7.4 (TBS). Endogenous peroxidase activity was blocked by incubating the section in 1.0% H2O2, 0.1% NaN₃ in TBS for 10 minutes. Sections were washed in TBS and subsequently incubated in 4% non-fat skim milk powder in TBS for 15 minutes. Sections were rinsed briefly in TBS and then incubated with 10% normal goat serum (NGS) for 20 minutes in a humidified chamber. Excess normal serum was decanted and primary antibody (or TBS as negative control) was applied overnight at room temperature. Sections were washed in TBS and then incubated with secondary antibody (DAKO, Glostrup Denmark, EnVision Kit) for 45 minutes. Sections were washed in TBS and colour was developed in 3,3-diaminobenzidine (DAB) with H₂O₂ as substrate. Sections were washed in gently running tap water then lightly counterstained in Mayers' haemotoxylin, dehydrated through ascending graded alcohols, cleared in xylene, and mounted in DPX mounting medium.

G3BP-2 is Over-Expressed in 88% of Breast Tumours

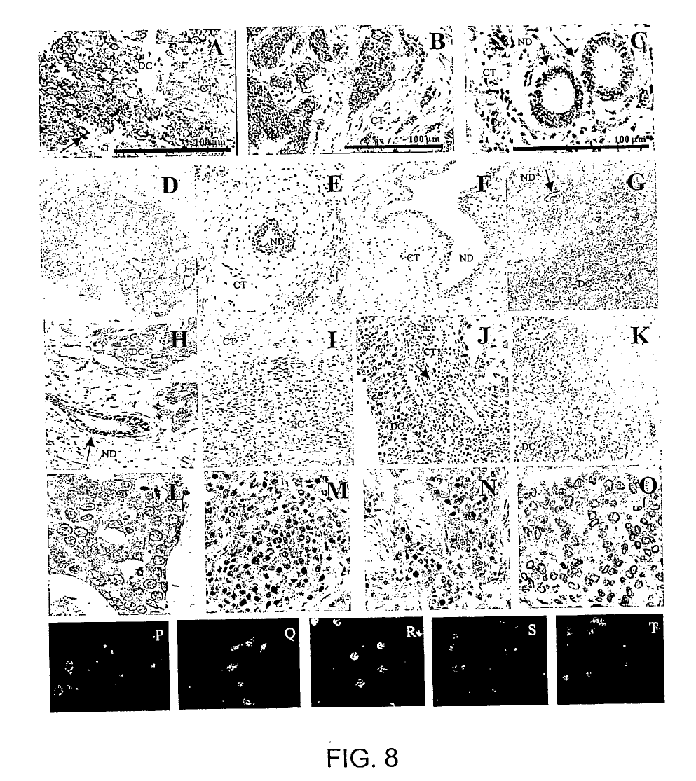

In addition to determining expression of G3BP-2, expression of G3BP-1 was also examined in 24 breast tumour cases by immunohistochemistry as shown in FIG. 8. Of these, 22 sections were infiltrating ductal carcinomas (IDC) and two were cases of infiltrating lobular carcinoma (ILC). All sections were counterstained with haematoxylin, which stains nuclei blue and the expression of G3BP-1 was visualised using horseradish peroxidase seen as brown staining (See FIG. 8, Panels A-C). Most normal cells exhibited detectable cytoplasmic expression of G3BP-1 (see FIG. 8, Panel C). Two normal ducts (ND) are seen in FIG. 8, Panel C, and cytoplasmic expression of G3BP-1 is apparent as seen by the distinct brown Panel A and B, but the adjacent connective tissue (CT) does not express G3BP-1 at detectable levels. The tumour cells in FIG. 8, Panels A and B appear to express higher levels of G3BP-1 in the cytoplasm as compared to that seen in the normal ducts of FIG. 8, Panel C.

In many cases tumour staining was heterogeneous and in some cases G3BP-1 appeared to localise more prominently to one side of the cell. This can be seen quite clearly in some of the tumour cells in FIG. 8, Panel A (indicated by the arrow). There was no nuclear staining present in any normal cells, although two of the 24 tumour cases contained distinct nuclear staining in less than 10% of tumour cells.

In summary, most normal breast cells expressed G3BP-1, but all of the tumours examined appeared to over-express G3BP-1 to some extent (see Table 3). No significant relationship was found between G3BP-1 over-expression and clinicopathological parameters of breast cancer such as lymph node involvement, hormone receptor status or nuclear or histological grade.

A total of 58 breast tumour cases were examined by immunohistochemistry for altered G3BP-2 expression. Of these, 54 tumours were IDCs and four were ILCs. As with G3BP-1, all sections were counterstained with haematoxylin and the expression of G3BP-2 was visualised using horseradish peroxidase (See FIG. 7 and FIG. 8, Panels D-O). Unlike that for G3BP-1, the immunohistochemistry showed no detectable expression of G3BP-2 in normal lobes of the breast (See FIG. 8, Panel D) including the lobular and ductal epithelium and surrounding connective tissue. Panels E and F of FIG. 8 show a higher magnification of two different ducts, Panel E shows a transverse section of a duct and Panel F shows a longitudinal section. As can be seen, there is no detectable expression of G3BP-2 in normal ducts of the breast or within cells of the surrounding connective tissue.

Immunohistochemistry revealed that G3BP-2 is over-expressed in breast tumours. FIG. 8, Panel G shows a normal duct adjacent to an IDC. As can be seen by the brown staining, G3BP-2 is highly expressed in the tumour but not expressed in the normal duct. This can be seen more clearly in Panel H which shows an IDC at higher magnification adjacent to a normal duct. Again, the normal duct does not express G3BP-2 and the IDC highly expresses G3BP-2.

The over-expression of G3BP-2 in breast tumours was not seen in all breast tumours examined (12%). Panel I of FIG. 8 shows one case of IDC that does not express G3BP-2. Another interesting observation noted when examining the expression of G3BP-2 in human breast tumours was that in some cases G3BP-2 is expressed in the nucleus of normal cells within the connective tissues lying between the tumours (marked by the arrow), but not in the cells within connective tissue away from the tumour (See FIG. 8, Panel J).

FIG. 8, Panel K shows an example of a lower magnification of a tumour and adjacent connective tissue. As can be seen, G3BP-2 is expressed in cells within the connective tissue peripheral to the tumour and the expression becomes lower the further away the cells are from the tumour. These cells are most likely infiltrating lymphocytes as there seems to be a greater population of these cells around the tumours. This could suggest that G3BP-2 expression is induced in response to a factor secreted by some tumours or that G3BP-2 produces a chemotaxis-like effect.

Table 2 shows the results of all breast tumours examined for G3BP-2 expression. Also listed is the available information on each of the breast tumours including its oestrogen receptor status, tumour grade and stage. In summary, 88% of all tumours examined over-express G3BP-2 and no significant relationship was found between G3BP-2 over-expression and clinicopathological parameters of breast cancer such as stage, hormone receptor status or nuclear or histological grade.

In the majority of human breast tumours that were screened, G3BP-2 is over-expressed and in many cases shows a distinct nuclear localisation (See FIG. 8, Panel M to O). Panels L to O show four different cases of IDC with three different sub-cellular localisations. Panel L is an example of a breast tumour where G3BP-2 is exclusively cytoplasmic. Panel M and N are two examples of breast tumours where G3BP-2 is found in the nucleus and in the cytoplasm. Panel O shows G3BP-2 localised around the nuclear envelope region. These tumours also show a cytoplasmic distribution for G3BP-2. This is the first case in which G3BP-2 has been found in the nucleus in situ. Approximately 50% of all tumours that express G3BP-2 have G3BP-2 in the nucleus, although it should be noted that it is possible that nuclear staining was not observed in some cells where G3BP-2 is expressed at low levels, due to masking by the haematoxylin counterstain. The nuclear staining varied between cases. Some cases had G3BP-2 in the nucleus of all cells, whereas others had less than 10% of cells with nuclear staining. The percentage of cells which express G3BP-2 in the nucleus does not correlate with the grade of the tumour or level of metastasis.

### EXAMPLE 9

G3PB-2 Expression During the Cell Cycle

Interestingly, G3BP-2 can shuttle into the nucleus and its movement appears to be cell cycle dependent (See FIG. 8P-8T). In serum starved resting cells G3BP-2 localises to the cytoplasm (FIG. 8P); however, within 2 hrs of releasing cells from G₀ G3BP-2 can be seen to move into the nucleus (FIG. 8Q-8T) and at 5 hrs appears to be almost totally nuclear (FIG. 8R). After this time G3BP can be seen in both compartments consistent with it shuttling between the nucleus and the cytoplasm (FIGS. 8S and 8T).

FIG. 8P-8T show the immunofluorescence of synchronised NIH 3T3 cells. Cells were synchronised by serum starvation and subsequently induced to enter the cell cycle by serum stimulation. Cells were stained for G3BP-2 using immunofluorescent technique at several time intervals following serum starvation. FIG. 8P shows the sub-cellular localisation of G3BP-2 in cells in G₀ phase (time=0). The time after serum stimulation and hence cell cycle commencement is 2 hours, 5 hours, 9 hours and 12 hours for FIGS. 8Q, 8R, 8S and 8T, respectively.

Cell Cycle Synchronisation of NIH 3T3 Cells

Cell cycle synchronisation of NIH 3T3 cells was performed using the serum deprivation method (Tobey et al. 1988). NIH 3T3 cells were seeded onto coverslips at sub-confluent conditions in 10% FCS. Following 24 hours, the cells were washed 3 times with PBS and serum free medium was then replaced with medium containing 10% FCS. Coverslips were removed from the media during serum starvation, and at 2, 5, 9 and 12 hours after serum stimulation. The coverslips were then processed for immunofluorescence to examine the expression of G3BP-2.

Immunofluorescence of Cultured Cells

NIH 3T3 cells were grown on coverslips, treated as described above, and washed 3×2 min with PBS and dried overnight at room temperature. The cells were fixed with 100% cold acetone for 5 min, allowed to dry, then rehydrated by washing the coverslips with PBS 3×5 min. The cells were then permeabilised by incubating the coverslips in 0.1% Triton-X 100 in PBS for 5 min. The detergent was then removed by washing the coverslips 3×5 min with PBS. Primary antibody, diluted to the appropriate concentration in PBS, was applied and left overnight at 4° C. The following morning the coverslips were washed 3×5 min in 1% normal goat serum (NGS)/1% bovine serum albumin (BSA) in PBS. Secondary antibody was then applied and incubated for 1 hour at room temperature. The secondary antibody was anti-rabbit IgG conjugated with either a FITC or Rhodamine fluorescent tag (Molecular Probes, Eugene, USA) and was diluted in 0.1% Triton-X 100 in PBS at the dilution specified by the manufacturer. The coverslips were then washed 2×5 min in 0.1% Triton-X 100 in PBS followed by 2×5 min PBS. Finally, coverslips were mounted onto slides with 50% glycerol/50% PBS and sealed with nail polish. Images were generated using an Olympus Provis AX-70 and captured in digital format with a DAGE-MTI CCD camera using Scion Image 1.62 frame grabber software. Images were analysed using Adobe Photoshop 5 image-processing software (Adobe systems incorporated, Eastman Kodak Company, 1996).

### EXAMPLE 10

Dendritic Cell Therapy

The invention provides pharmaceutical compositions and methods for preventing or treating breast cancer in an animal. The pharmaceutical composition comprises an isolated antigen presenting cell which has been in contact with G3BP-2 polypeptide, fragment, homolog or derivative thereof, thereby loading or pulsing the cell with antigen. The isolated antigen presenting cell is preferably a dendritic cell isolated from a patient which is undergoing dendritic cell therapy. The antigen presenting cell may also be a precursor dendritic cell. The antigen presenting cell may be cultured in vitro to expand or increase the number of cells before or after antigen loading. Alternatively, or in addition, to loading the antigen presenting cell with G3BP-2 polypeptide, fragment, homolog or derivative thereof, the antigen presenting cell may be transfected with a nucleic acid encoding G3BP-2 polypeptide, fragment, homolog or derivative thereof. The nucleic acid may be DNA or RNA.

The method of preventing or treating breast cancer in an animal includes the step of administering to the animal a pharmaceutical composition comprising antigen presenting cells which have been loaded or pulsed with G3BP-2 polypeptide, fragment, homolog, or derivative thereof; or cells which have been transfected with a nucleic acid encoding G3BP-2, fragment, homolog, variant or derivative thereof.

In one embodiment, the method includes the steps of: (a) isolating antigen presenting cells from an animal; (b) contacting the isolated cells with G3PB-2 polypeptide, fragment, homolog, or derivative thereof, thereby antigen loading or pulsing the isolated cells; and (c) administering the loaded or pulsed isolated cells to the animal. The cells are preferably autologous dendritic cells isolated from an animal which is administered the pharmaceutical composition.

In another embodiment, the invention method includes the steps of: (a) isolating antigen presenting cells from an animal; (b) transfecting the isolated cells with a nucleic acid encoding G3PB-2 polypeptide, fragment, homolog, or derivative thereof; and (c) administering the transfected cells to the animal. The cells are preferably autologous dendritic cells isolated from an animal which is administered the pharmaceutical composition.

In either or both embodiments, the method may further include the step of expanding the isolated antigen presenting cells in culture before step (c).

G3BP-2 and New Immunotherapy for Breast Cancer

Immuno-prevention is a very attractive therapy for breast cancer due to the minimal tumor load and an ideal target for the intervention by the immune system. One of the most promising strategies for immuno-prophylaxic therapy is based on the use of dendritic cells (DC), the most potent antigen presenting cell (APC) of the immune system, responsible for the initiation of the immune response (Hart, 1997) and vital link between the innate and adaptive immunity (Clark et al., 2000). Researchers have investigated the important role of DC in health and disease (Ho et al., 2001) and established mechanisms to optimize their APC capacity (Ho et al., 2002). Their potent capacity to induce cytotoxic T-lymphocyte (CTL) responses has been harnessed with success for the treatment of various tumors (reviewed in (Lopez and Hart, 2002)). Furthermore, it has been demonstrated that it is possible to obtain large number of DC from the blood (Lopez et al., 2002). This very promising field of research still awaits critical inputs before being applied in a generic form.

In breast cancer research, the most important hurdle is the availability of tumor-associated antigens (TAA) that may elicit strong enough immune responses. An ideal TAA would have the characteristics of being crucial for the development of cancer cells, over-(or selectively) expressed in cancer cells, intracellularly localized and recognized by the immune system (Pardoll, 2002). Only two firm TAA candidates are available and they are currently being tested. MUC-1, (CD227) is a transmembrane mucin molecule normally polarised to the apical surface of epithelial cells characterized by a large extracellular domain of GC-rich random repeats (Gendler et al., 1991); it is highly expressed in breast cancer cells and over-expressed in more than 90% of patients with breast cancer (Hadden, 1999). Her2/neu is a transmembrane glycoprotein, homologous to the epidermal growth factor receptor that is over expressed in 20-30% of patients with breast cancer (Wang et al., 2001). It is correlated to the aggressiveness of the disease and an indicator of poor prognosis. Both cellular and humoral immune responses to this protein have been detected in patients (Wang and Hung, 2001). Additionally, TAA shared with other malignancies, eg. MAGE 1 and 3 (melanoma antigens) are expressed in 20% and 26% of breast cancers respectively (Mashino et al., 2001; Otte et al., 2001; Russo et al., 1995). Finally, newer antigens such as the carbohydrate antigen globo H (Gilewski et al., 2001) have emerged lately, and are currently being evaluated. None of the available TAA candidates fulfill the abovementioned criteria for an optimal TAA in breast cancer. The present researcher's recent data, however, indicate that G3BP proteins represent excellent candidate TAA.

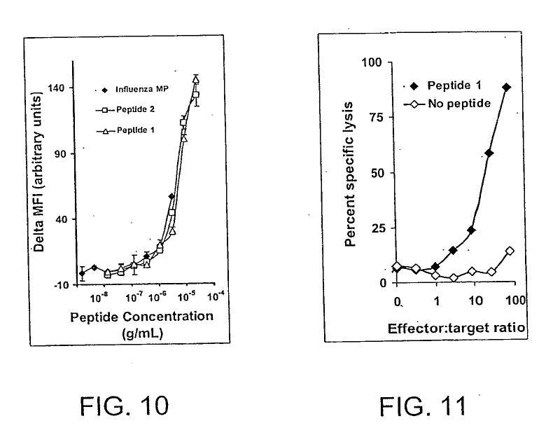

Immunogenicity of G3BP proteins within the context of the HLA-A*0201 molecule have been evaluated, the most commonly found allele of the Major Histocompatibility Complex (MHC), following the genomic/immunogenic approach (Schultze and Vonderheide, 2001), incorporated herein by reference. Predicted HLA binding sequences were identified applying web-based algorithms (for example as provided by SYPEITHI (http://svfpeithi.bmi-heidelberg.com/) and BIMAS (http://bimas.dcrt.nih.gov/molbio/hla_bind/) and synthetic peptides produced for further evaluation. From the peptides generated, MHC/peptide binding assays demonstrated a very strong binding to the HLA-A*0201 molecule, as shown in FIG. 10. T2 cells were incubated with peptide dilutions shown and HLA expression measured by flow cytometry. Two peptides (peptide 1 and 2) of G3BP-2 were tested and control matrix protein influenza peptide 58-66 was used as a reference. Peptide 1 has an amino acid sequence of KLPNFGFVV [SEQ ID NO:1] and peptide 2 has an amino acid sequence of IMFRGEVRL [SEQ ID NO:2]. Peptides 1 and 2 bound to the HLA-A*0201 molecule with an affinity equal to that of the control influenza MP 58-66 peptide, a well-defined CTL epitope.

The various peptides were evaluated for the generation of CTL responses induced by DC in peripheral blood mononuclear cells (PBMC) from healthy individuals and tested in ELISPOT (Gonzalez et al, 2000). T2 cells (target) pulsed with Peptide 1 or no peptide were labelled with ⁵¹Cr and incubated with clone IH7 (effector) at various ratios. After 4 hours, ⁵¹Cr released from lysed cells was measured and the percentage of specific lysis calculated with the formula: 100×(experimental release−spontaneous release)/(total release−spontaneous release). Total release was obtained with detergent lysis of labelled target.

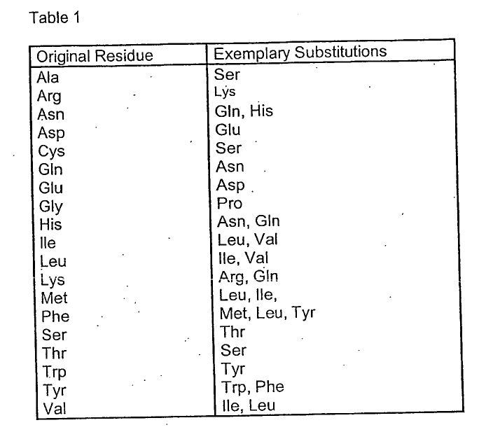

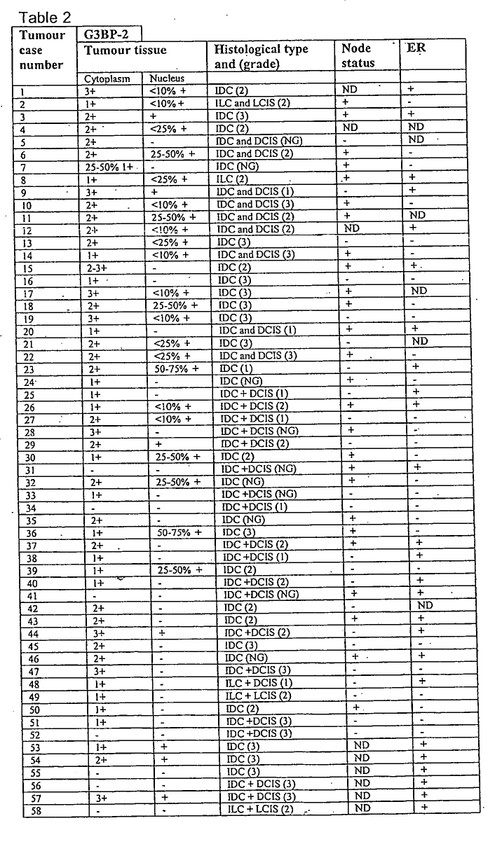

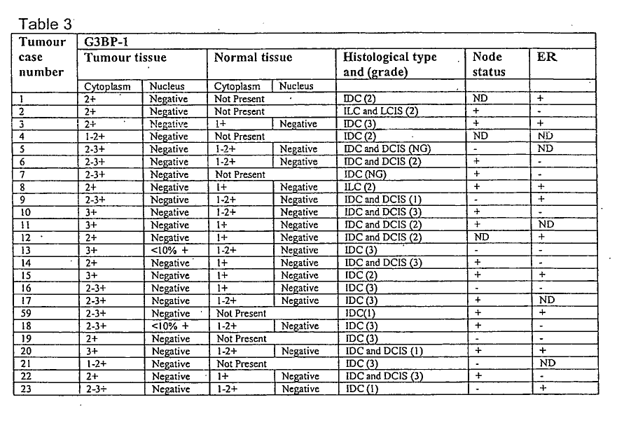

Strong responses were efficiently generated for Peptide 1, demonstrated by high frequencies of IFN-γ producing peptide specific CD8+ lymphocytes obtained after one (and two) round of stimulation in 2 separate donors. These results indicate that, indeed, the Peptide 1 epitope is included in the T-cell receptor (TCR) repertoire and is, therefore, immunogenic. Confirming this finding, we generated Peptide 1 specific CTL clones capable of identifying peptide-loaded targets in a classical chromium release assay (FIG. 11), the desired profile for an efficient anti-tumor response (Lopez and Hart, 2002), incorporated herein by reference. These clones will be instrumental in the evaluation of the expression of this antigen in cancer cells, allowing the formal evaluation of this molecule as a TAA.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

The disclosure of each patent and scientific document, computer program and algorithm referred to in this specification is incorporated by reference in its entirety.

### REFERENCES

1. (AIHW), A.I.o.H.a.W. (1999) Breast cancer in Australian women 1982-1996. Centre, A.A.o.C.R.N.N.B.C. (ed). Can berra.
2. Atkin N B. 1998. Cytogenetics of Hodgkin's disease. Cytogenet Cell Genet 80:23-7.
3. Birney E, Kumar S, Krainer A R. 1993. Analysis of the RNA-recognition motif and RS and RGG domains: conservation in metazoan pre-mRNA splicing factors. Nucleic Acids Research 21:5803-5816.
4. Burd C G, Dreyfuss G. 1994. Conserved structures and diversity of functions of RNA-binding proteins. Science 265:615-621.
5. Chlebowski, R. T. (2002) Breast cancer risk reduction: strategies for women at increased risk. Annu Rev Med 53: 519-540.
6. Clark, G. J., Angel, N., Kato, M., Lopez, J. A., Macdonald, K., Vuckovic, S., and Hart, D. N. J. (2000) The role of dendritic cells in the innate immune system. Microbes and Infection 2: 257-272.
7. Costa M, Ochem A, Staub A, Falaschi A. 1999. Human DNA helicase VIII: a DNA and RNA helicase corresponding to the G3BP protein, an element of the Ras transduction pathway. Nucl. Acids Res. 27:817-821.
8. Dreyfuss G, Hentze M, Lamond A I. 1996. From transcript to protein. Cell 85:963-972.
9. Duchesne M, Schweighoffer F, Parker F, Clerc F, Frobert Y, Thang M N, Tocque B. 1993. Identification of the SH3 domain of GAP as an essential sequence for Ras-GAP-mediated signaling. Science 259:525-8.
10. Gallouzi I E, Parker F, Chebli K, Maurier F, Labourier E, Barlat I, Capony J P, Tocque B, Tazi J. 1998. A novel phosphorylation-dependent RNase activity of GAP-SH3 binding protein: a potential link between signal transduction and RNA stability. Molec. Cell. Biol. 18:3956-3965.
11. Gendler, S. J., Spicer, A. P., Lalani, E. N., Duhig, T., Peat, N., Burchell, J., Pemberton, L., Boshell, M., and Taylor-Papadimitriou, J. (1991) Structure and biology of a carcinoma-associated mucin, MUC1. Am Rev Respir Dis 144: S42-47.
12. Gilewski, T., Ragupathi, G., Bhuta, S., Williams, L. J., Musselli, C., Zhang, X. F., Bornmann, W. G., Spassova, M., Bencsath, K. P., Panageas, K. S., Chin, J., Hudis, C. A., Norton, L., Houghton, A. N., Livingston, P. O., and Danishefsky, S. J. (2001) Immunization of metastatic breast cancer patients with a fully synthetic globo H conjugate: a phase I trial. Proc Natl Acad Sci USA 98: 3270-3275.
13. Gonzalez, J. M., Peter, K., Esposito, F., Nebie, I., Tiercy, J. M., Bonelo, A., Arevalo-Herrera, M., Valmori, D., Romero, P., Herrera, S., Corradin, G., and Lopez, J. A. (2000) HLA-A*0201 restricted CD8+ T-lymphocyte responses to malaria: identification of new Plasmodium falciparum epitopes by IFN-gamma ELISPOT. Parasite Immunol 22: 501-514.
14. Hadden, J. W. (1999) The immunology and immunotherapy of breast cancer: an update. Int J Immunopharmacol 21: 79-101.
15. Hart, D. N. J. (1997) Dendritic cells: unique leucocyte populations which control the primary immune response. Blood 90: 3245-3287.
16. Harlow E, Lane D (1988): Antibodies a laboratory manual New York: Cold Spring Harbor Laboratory.
17. Ho, C. S. K., Lopez, J. A., Vuckovic, S., Pyke, C. M., Hockey, R. L., and Hart, D. N. J. (2001) Surgical and physical stress increase circulating blood dendritic cell counts independently of monocyte counts. Blood 98: 140-145.
18. Ho, C. S. K., Munster, D., Pyke, C. M., Hart, D. N. J., and Lopez, J. A. (2002) Spontaneous generation and survival of blood dendritic cells in mononuclear cell culture without exogenous cytokines. Blood 99:2897-2904.
19. Horrigan S K, Bartoloni L, Speer M C, Fulton N, Kravarusic J, Ramesar R, Vance J M, Yamaoka L H, Westbrook C A. 1999. A radiation hybrid breakpoint map of the acute myeloid leukemia (AML) and limb-girdle muscular dystrophy 1A (LGMD1A) regions of chromosome 5q31 localizing 122 expressed sequences. Genomics 57:24-35.
20. Hu K-Q, Settleman J. 1997. Tandem SH2 binding sites mediate the RasGAP-RhoGAP interaction: a conformational mechanism for SH3 domain regulation. EMBO. J. 16:473-483.
21. Kane, D. A., Maischein, H. M., Brand, M., van Eeden, F. J., Furutani-Seiki, M., Granato, M., Haffter, P., Hammerschmidt, M., Heisenberg, C. P., Jiang, Y. J., Kelsh, R. N., Mullins, M. C., Odenthal, J., Warga, R. M., and Nusslein-Volhard, C. (1996) The zebrafish early arrest mutants. Development 123: 57-66.
22. Kennedy D, French J, Guitard E, Ru K, Tocque B, Mattick J (2001) Characterization of G3BPs: tissue specific expression, chromosomal localisation and rasGAP(120) binding studies. J Cell Biochem 84: 173-87.
23. Kennedy D, Wood S A, Ramsdale T, Tam P P, Steiner K, Mattick J S. 1997. Identification of a mouse orthologue of the human ras-GAP-SH3-domain binding protein and structural confirmation that these proteins contain an RNA recognition motif. Biomedical. Peptides, Proteins and Nucleic Acids 2:93-99.
24. Lee C H, Saksela K, Mirza U A, Chait B T, Kuriyan J. 1996. Crystal structure of the conserved core of HIV-1 Nef complexed with a Src family SH3 domain. Cell 85:931-42.
25. Liu Y F, Deth R C, Devys D. 1997. SH3 domain-dependent asscociation of Huntingtin with epidermal growth factor receptor signaling complexes. J. Biol. Chem. 272:8121-8124.
26. Lopez, J. A., and Hart, D. N. (2002) Current issues in dendritic cell cancer immunotherapy. Curr Opin Mol Ther 4: 54-63.
27. Macara I G. 1999. Nuclear transport: randy couples. Curr Biol 9:R436-9.
28. Macara, I. G. (2001) Transport into and out of the nucleus. Microbiol Mol Biol Rev 65: 570-594. Nagai K, Oubridge C, Ito N, Avis J, Evans P. 1995. The RNP domain: a sequence-specific RNA-binding domain involved in processing and transport of RNA. Trends Biochem. Sci. 20:235-240.
29. Mashino, K., Sadanaga, N., Tanaka, F., Yamaguchi, H., Nagashima, H., Inoue, H., Sugimachi, K., and Mori, M. (2001) Expression of multiple cancer-testis antigen genes in gastrointestinal and breast carcinomas. Br J Cancer 85: 713-720.
30. McGuckin M A, Walsh M D, Hohn B G, Ward B G, Wright R G (1995) Prognostic significance of MUC1 epithelial mucin expression in breast cancer. Hum Pathol 26: 432-9.
31. Nathanson, K. L., Wooster, R., Weber, B. L., and Nathanson, K. N. (2001) Breast cancer genetics: what we know and what we need. Nat Med 7: 552-556.
32. Otte, M., Zafrakas, M., Riethdorf, L., Pichimeier, U., Loning, T., Janicke, F., and Pantel, K. (2001) MAGE-A gene expression pattern in primary breast cancer. Cancer Res 61: 6682-6687.
33. Parker F Maurier F, Delumeau I, Duchesne M, Faucher D, Debussche L, Dugue A, Schweighoffer F, Tocque B. 1996. A ras-GTPase-activated protein SH3-domain-binding protein. Molecular and Cellular Biology 16:2561-2569.
34. Pazman C, Mayes C A, Fanto M, Haynes S R, Mlodzik M. 2000. Rasputin, the Drosophila homologue of the RasGAP SH3 binding protein, functions in ras- and Rho-mediated signaling. Development 127:1715-25.
35. Pardoll, D. M. (2002) Spinning molecular immunology into successful immunotherapy. Nat Rev Immunol 2: 227-238.
36. Pazman, C., Mayes, C. A., Fanto, M., Haynes, S. R., and Mlodzik, M. (2000) Rasputin, the Drosophila homologue of the RasGAP SH3 binding protein, functions in ras- and Rho-mediated signaling. Development 127: 1715-1725.
37. Pomerance M, Thang M N, Tocque B, Pierre M. 1996. The ras-GTPase-activating protein SH3 domain is required for Cdc2 activation and Mos induction by oncogenic ras in Xenopus oocytes independently of mitogen-activated protein kinase activation. Molecular and Cellular Biology 16:3179-3186.
38. Prigent M, Baarlat I, Langen H, Dargemont C 2000. IκBα and IκBα/NT-KB complexes are retained in the cytoplasm through interactions with a novel partner, rasGAP SH3-binding protein 2. Jour. Biol. Chem. 275: 36441-36449.
39. Pritchard, K. I., Khan, H., and Levine, M. (2002) Clinical practice guidelines for the care and treatment of breast cancer: 14. The role of hormone replacement therapy in women with a previous diagnosis of breast cancer. Cmaj 166: 1017-1022.
40. Russo, V., Traversari, C., Verrecchia, A., Mottolese, M., Natali, P. G., and Bordignon, C. (1995) Expression of the MAGE gene family in primary and metastatic human breast cancer: implications for tumor antigen-specific immunotherapy. Int J Cancer 64: 216-221.
41. Sambrook J, Fritsch E F, Maniatis T 1989. Molecular Cloning A Laboratory Manual. Cold Spring Harbor: Cold Spring Harbor Laboratory Press.
42. Schultze, J. L., and Vonderheide, R. H. (2001) From cancer genomics to cancer immunotherapy: toward second-generation tumor antigens. Trends Immunol 22: 516-523.
43. Siomi H, Dreyfuss G. 1997. RNA-binding proteins as regulators of gene expression. Current Opinion in Genetics and Development 7:345-353.
44. Suyama M, Doerks T, Braun I C, Sattler M, Izaurralde E, Bork P. 2000. Prediction of structural domains of TAP reveals details of its interaction with p15 and nucleoporins. EMBO rep 1:53-8.
45. Svitkin Y V, Ovchinnikov L P, Dreyfuss G, Sonenberg N. 1996. General RNA binding proteins render translation cap dependent. EMBO. J. 15:7147-7155.
46. Tocque B, Delumeau I, Parker F, Maurier F, Multon C-M, Schweighoffer F. 1997. Ras-GTPase activating protein (GAP): A putative effector for ras. Cell. Signal. 9:153-158.
47. Tobey R A, Valdez J G, Crissman H A (1988) Synchronization of human diploid fibroblasts at multiple stages of the cell cycle. Exp Cell Res 179: 400-16.
48. Urquhart A J, Kennedy D, Gould S J, Crane D I. 2000. Interaction of Pex5p, the Type 1 Peroxisome Targeting Signal Receptor, with the Peroxisomal Membrane Proteins Pex14p and Pex13p. J. Biol. Chem. 275:4127-4136.
49. Wang, S. C., and Hung, M. C. (2001) HER2 overexpression and cancer targeting. Semin Oncol 28: 115-124.
50. Wang, S. C., Zhang, L., Hortobagyi, G. N., and Hung, M. C. (2001) Targeting HER2: recent developments and future directions for breast cancer patients. Semin Oncol 28: 21-29.
51. Wong N, Lai P, Lee S W, Fan S, Pang E, Liew C T, Sheng Z, Lau J W, Johnson P J. 1999. Assessment of genetic changes in hepatocellular carcinoma by comparative genomic hybridization analysis: relationship to disease stage, tumor size, and cirrhosis. Am J Pathol 154:37-43.

1. An isolated G3BP-2 protein fragment comprising an NTF2-like domain, said isolated G3BP-2 protein fragment capable of binding another protein by way of said NTF2-like domain.

2. The isolated G3BP-2 protein fragment of claim 1 wherein the isolated G3BP-2 protein fragment comprises isolated G3BP-2a protein fragment and/or G3BP-2b protein fragment.

3. The isolated G3BP-2 protein fragment of claim 1 wherein said another protein is selected from the group consisting of: ran nuclear pore polypeptide, ubiquitin hydrolase and GAP¹²⁰.

4. The isolated G3BP-2 protein fragment of claim 3 wherein the ubiquitin hydrolase is ODE1.

5. The isolated G3BP-2 protein fragment of claim 1 wherein the NTF2-like domain comprises an amino acid sequence as set forth in SEQ ID NO: 22.

6. An isolated protein complex comprising a G3BP-2 protein and a second protein bound to an NTF2-like domain of the G3BP-2 protein.

7. The isolated protein complex of claim 6 wherein said second protein is selected from the group consisting of: ran nuclear pore polypeptide, ubiquitin hydrolase and GAP¹²⁰.

8. An isolated G3BP-2 protein, inclusive of a fragment, homolog, variant or derivative used to elicit an immune response in an animal.

9. The isolated G3BP-2 protein of claim 8 wherein said animal is human.

10. The isolated G3BP-2 protein of claim 8 selected from the group

| | | |
|---|---|---|
| (i) | KLPNFGFW; | [SEQ ID NO:1] |
| (ii) | IMFRGEVRL; | [SEQ ID NO:2] |
| and | | |
| (iii) | SATPPPAEPASLPQEPPKPRV | [SEQ ID NO:3] |

11. An isolated G3BP-2 protein fragment selected from the group consisting of:

| | | |
|---|---|---|
| (a) | KLPNFGFW; | [SEQ ID NO:1] |
| (b) | IMFRGEVRL; | [SEQ ID NO:2] |
| and | | |
| (c) | SATPPPAEPASLPQEPPKPRV | [SEQ ID NO:3] |

12. An isolated nucleic acid encoding a protein of claim 1, inclusive of fragments, homologs, variants and derivatives of said isolated protein.

13. The isolated nucleic acid of claim 12 encoding a protein comprising the NTF2-like domain comprising an amino acid sequence as set forth in SEQ ID NO: 22.

14. The isolated nucleic acid of claim 12 comprising a sequence set forth in SEQ ID NO: 23.

15. An isolated nucleic acid encoding the G3BP-2 protein fragment of claim 10.

16. An expression vector comprising the nucleic acid of claim 12 or claim 14.

17. Use of an antagonist to prevent or disrupt binding between G3BP-2 and another protein.

18. Use of the antagonist of claim 17, whereby said antagonist prevents or disrupts binding between a NTF2-like domain of G3BP-2 and said another protein.

19. Use of the antagonist of claim 17 wherein said antagonist is a mimetic of the NTF2-like domain of G3BP-2.

20. Use of the antagonist of claim 17 wherein said antagonist binds to the NTF2-like domain.

21. Use of the antagonist of claim 17 wherein said antagonist is a protein.

22. Use of the antagonist of claim 21 wherein said protein comprises an Src homology 3 (SH3) domain.

23. Use of the antagonist of claim 22 wherein said protein comprises an amino acid sequence as set forth in SEQ ID NO: 6.

24. Use of the antagonist of claim 17 wherein said antagonist is a non-peptide compound.

25. An isolated antigen presenting cell which has been contacted with a G3BP-2 protein, fragment, homolog, variant or derivative thereof.

26. An isolated antigen presenting cell which has been transfected with a nucleic acid encoding a G3BP-2 protein, inclusive of fragments, homologs, variants and derivatives thereof.

27. The isolated antigen presenting cell of claim 25 or claim 26 wherein said cell is a dendritic cell.

28. The isolated antigen presenting cell of claim 25 or claim 26 wherein said G3BP-2 protein, inclusive of a fragment, a homolog, a variant and a derivative thereof comprises an amino acid sequence as set forth in SEQ ID NO: 5.

29. The isolated antigen presenting cell of claim 25 or claim 26 wherein said G3BP-2 fragment comprises an amino acid sequence selected from the group consisting of: KLPNFGFVV [SEQ ID NO: 1] and IMFRGEVRL [SEQ ID NO: 2].

30. An isolated lymphocyte that is G3BP-2 antigen specific.

31. The isolated lymphocyte of claim 30, wherein said isolated lymphocyte is a cytotoxic T-lymphocyte.

32. The isolated lymphocyte cell of claim 30, wherein said G3BP-2 antigen is a protein, inclusive of fragments, homologs, variants and derivatives thereof, comprises an amino acid sequence as set forth in SEQ ID NO: 5.

33. The isolated lymphocyte of claim 30, wherein said G3BP-2 protein fragment comprises an amino acid sequence as set forth in SEQ ID NO: 1 or SEQ ID NO: 2.

34. A pharmaceutical composition comprising at least one active, wherein the active is selected from the group consisting of: a protein of any one of claims 1, 6, 11; a nucleic acid of any one of claims 12, 15, 16 or an isolated antigen presenting cell or lymphocyte of any one of claims 25, 26, 30.

35. A method for preventing or treating breast cancer in a mammal including the step of administering to said mammal a pharmaceutical composition comprising at least one active, wherein the active is selected from the group consisting of: a protein of any one of claims 1, 6, 11; a nucleic acid of any one of claims 12, 15, 16, a mimetic of the NTF2-like domain of G3BP-2; an antagonist that prevents or disrupts binding between a NTF2-like domain of G3BP-2 and another protein; or isolated cell of any one of claims 25, 26, 30.

36. The method of claim 35 wherein said mammal is human.

37. A method for modulating cell proliferation including the step of administering to an animal or isolated cell, an active which prevents or disrupts binding between G3BP-2 and another protein.

38. The method of claim 37 wherein said animal is human.

39. A method for isolating a molecule that binds G3BP-2, including the step of determining if one or more candidates in a sample bind to the NTF2-like domain of G3BP-2.

40. The method of claim 39 wherein said molecule is an antagonist.

41. The method of claim 40 wherein said antagonist is a protein or a non-protein molecule.

42. A method for diagnosing breast cancer in a mammal including the steps of comparing G3BP-2 protein expression in a test sample obtained from the mammal with G3BP-2 in a reference sample, wherein if the expression of G3BP-2 in the test sample is different than the reference sample, the mammal is diagnosed with an increased likelihood of having breast cancer.

43. The method of claim 42 when G3BP-2 protein expression is detected using an antibody.

44. The method of claim 43 wherein said antibody binds to a G3BP-2 protein, inclusive of a fragment, a homolog, a variant and a derivative thereof, comprising an amino acid sequence as set forth in SEQ ID NO: 5.

45. The method of claim 44 wherein said G3BP-2 fragment comprises a NTF2-like domain.

46. The method of claim 43 wherein said antibody binds to a G3BP-2 protein fragment comprising an amino acid sequence SATPPPAEPASLPQEPPKPRV [SEQ ID NO: 3].

47. The method of claim 42 wherein said mammal is human.

48. The method of claim 48 wherein said test sample is breast tissue.

49. A method for diagnosing breast cancer in a mammal including the step of detecting a G3BP-2 nucleic acid or fragment thereof in a test sample obtained from the mammal.

50. The method of claim 48 wherein said mammal is human.

51. A method of immunising a mammal against breast cancer, including the step of administering to said mammal an immunogenic agent comprising at least one active selected from the group consisting of:
(1) a G3BP-2 protein;
(2) a fragment, a homolog, a variant or a derivative of (1);
(3) a G3BP-2 nucleic acid;
(4) a fragment, a homolog, a variant or a derivative of (3);
(5) an isolated antigen presenting cell that has been contacted with (1) or (2); and
(6) an isolated antigen presenting cell that has been transfected with a nucleic acid of (3) or (4).

52. The method of claim 51 wherein said G3BP-2 protein fragment is selected from the group consisting of: KLPNFGFVV [SEQ ID NO: 1] and IMFRGEVRL [SEQ ID NO: 2].

53. The method of claim 51 wherein said antigen presenting cell is a dendritic cell.

54. The method of claim 51 wherein said mammal is human.
